SEQUENCE LISTING (90 sequences)

SEQ ID NO: 1 - DNA, 444 bp, Homo sapiens
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaacc

SEQ ID NO: 2 - protein, 148 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQT

SEQ ID NO: 3 - DNA, 354 bp, Mus musculus
caggtgcagctgaaggagtcaggacctggcctggtggcgccctcaaagaacctgtccatcacttgcactgtctctgggttttcattaaccagctatggtgtacactggattcgccagcctccaggaaagggactggaatggctgggagtaatatgggccggtggaagcacaaattataattcggctctcatgtccagactgagcatcagcaaagacaactccaggagccaagttttcttaagaatgaacagtctgcaaactgatgacacagccatgtactactgtgccagagcctatggtaactactggtacatcgatgtctggggcgcagggaccacggtcaccgtctcctca

SEQ ID NO: 4 - protein, 118 aa, Mus musculus
QVQLKESGPGLVAPSKNLSITCTVSGFSLTSYGVHWIRQPPGKGLEWLGVIWAGGSTNYNSALMSRLSISKDNSRSQVFLRMNSLQTDDTAMYYCARAYGNYWYIDVWGAGTTVTVSS

SEQ ID NO: 5 - DNA, 321 bp, Mus musculus
gacattgtgatgacccagactcccaaattcctgcttgtatcagcaggagacagggttaccataacctgcaaggccagtcagagtgtgagtaatgatgtagcttggtaccaacagaagccagggcagtctcctaaactgctgataaactatgcatttcatcgcttcactggagtccctgatcgtttcactggcagtggatatgggacggatttcattttcaccatcagcactgtgcaggctgaagacctggcagtttatttctgtcaccaggcttatagttctccgtacacgttcggaggggggaccaagctggaaatgaaa

SEQ ID NO: 6 - protein, 107 aa, Mus musculus
DIVMTQTPKFLLVSAGDRVTITCKASQSVSNDVAWYQQKPGQSPKLLINYAFHRFTGVPDRFTGSGYGTDFIFTISTVQAEDLAVYFCHQAYSSPYTFGGGTKLEMK

SEQ ID NO: 7 - DNA, 342 bp, Mus musculus
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaataataatggagagccaacatatgctgaagagttcaagggacggtttgccttctctttggaaacctctgccagcactgcctatttgcagatcaacaacctcaaaaatgaggacacggctacatatttctgtgcaagagatgttatggactattggggtcaaggaacctcagtcaccgtctcctca

SEQ ID NO: 8 - protein, 114 aa, Mus musculus
QIQLVQSGPELKKPGETVKISCKASGYTFTNYGMNWVKQAPGKGLKWMGWINNNNGEPTYAEEFKGRFAFSLETSASTAYLQINNLKNEDTATYFCARDVMDYWGQGTSVTVSS

SEQ ID NO: 9 - DNA, 330 bp, Mus musculus
gacattgtgctgacccaatctccagcttctttggctgtgtctctagggcagagggccaccatatcctgcagagccagtgaaagtgttgataattatggctatagttttatgcactggtaccagcagaaaccaggacagccaccccaactcctcatctatcgtgcatccaacctagaatctgggatccctgccaggttcagtggcagtgggtctaggacaggcttcaccctcaccattaatcctgtggaggctgatgatgttgcaacctattactgtcagcaaagtaaagaatatccgacgttcggtggaggcaccaagctggaagtcaaa

SEQ ID NO: 10 - protein, 110 aa, Mus musculus
DIVLTQSPASLAVSLGQRATISCRASESVDNYGYSFMHWYQQKPGQPPQLLIYRASNLESGIPARFSGSGSRTGFTLTINPVEADDVATYYCQQSKEYPTFGGGTKLEVK

SEQ ID NO: 11 - protein, 10 aa, Mus musculus
GFSLTSYGVH

SEQ ID NO: 12 - protein, 16 aa, Mus musculus
VIWAGGSTNYNSALMS

SEQ ID NO: 13 - protein, 12 aa, Mus musculus
ARAYGNYWYIDV

SEQ ID NO: 14 - protein, 11 aa, Mus musculus
KASQSVSNDVA

SEQ ID NO: 15 - protein, 7 aa, Mus musculus
YAFHRFT

SEQ ID NO: 16 - protein, 9 aa, Mus musculus
HQAYSSPYT

SEQ ID NO: 17 - protein, 10 aa, Mus musculus
GYTFTNYGMN

SEQ ID NO: 18 - protein, 17 aa, Mus musculus
WINNNNGEPTYAEEFKG

SEQ ID NO: 19 - protein, 7 aa, Mus musculus
ARDVMDY

SEQ ID NO: 20 - protein, 15 aa, Mus musculus
RASESVDNYGYSFMH

SEQ ID NO: 21 - protein, 7 aa, Mus musculus
RASNLES

SEQ ID NO: 22 - protein, 8 aa, Mus musculus
QQSKEYPT

SEQ ID NO: 23 - DNA, 354 bp, Artificial Sequence
  317-4B6 cDNA-Vh
caggtgcagctgcaggagtcgggaccaggactggtgaagccttcggagaccctgtccctcacctgcactgtctctgggttttcattaaccagctatggtgtacactggatccggcagcccccagggaagggactggagtggatcggggtcatatacgccgatggaagcacaaattataatccctccctcaagagtcgagtgaccatatcaaaagacacctccaagaaccaggtttccctgaagctgagctctgtgaccgctgcggacacggccgtgtattactgtgcgagagcctatggtaactactggtacatcgatgtctggggccaagggaccacggtcaccgtctcctca

SEQ ID NO: 24 - protein, 118 aa, Artificial Sequence
  317-4B6 pro-Vh
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 25 - DNA, 321 bp, Artificial Sequence
  317-4B6 cDNA-Vk
gacatcgtgatgacccagtctccagactccctggctgtgtctctgggcgagagggccaccatcaactgcaagtccagcgagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggccaggggaccaagctggagatcaaa

SEQ ID NO: 26 - protein, 107 aa, Artificial Sequence
  317-4B6 pro-Vk
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 27 - DNA, 342 bp, Artificial Sequence
  326-4A3 cDNA-Vh
caggtgcagctggtgcagagcggcagcgagctgaagaagcccggcgccagcgtgaaggtgagctgcaaggccagcggctacaccttcaccaactacggcatgaactgggtgagacaggcccccggccagggcctgaagtggatgggctggatcaacaacaacaacgccgagcccacctacgcccaggacttcagaggcagattcgtgttcagcctggacaccagcgccagcaccgcctacctgcagatcagcagcctgaagaccgaggacaccgccgtgtactactgcgccagagacgtgatggactactggggccagggcaccctggtgaccgtgagcagc

SEQ ID NO: 28 - protein, 114 aa, Artificial Sequence
  326-4A3 pro-Vh
QVQLVQSGSELKKPGASVKVSCKASGYTFTNYGMNWVRQAPGQGLKWMGWINNNNAEPTYAQDFRGRFVFSLDTSASTAYLQISSLKTEDTAVYYCARDVMDYWGQGTLVTVSS

SEQ ID NO: 29 - DNA, 330 bp, Artificial Sequence
  326-4A3 cDNA-Vk
gacattgtgctgacccagtctccagcctccttggccgtgtctccaggacagagggccaccatcacctgcagagccagtgaaagtgttgataattatggctatagttttatgcactggtatcagcagaaaccaggacaacctcctaaactcctgatttaccgtgcatccaacctagaatctggggtcccagccaggttcagcggcagtgggtctgggaccgatttcaccctcacaattaatcctgtggaagctgaggatactgcaaattattactgtcagcaaagtaaagaatatccgacgttcggcggagggaccaaggtggagatcaaa

SEQ ID NO: 30 - protein, 110 aa, Artificial Sequence
  326-4A3 pro-Vk
DIVLTQSPASLAVSPGQRATITCRASESVDNYGYSFMHWYQQKPGQPPKLLIYRASNLESGVPARFSGSGSGTDFTLTINPVEAEDTANYYCQQSKEYPTFGGGTKVEIK

SEQ ID NO: 31 - protein, 10 aa, Mus musculus
GFSLTSYGVH

SEQ ID NO: 32 - protein, 16 aa, Artificial Sequence
  317-4B6 H-CDR2 or CDR-H2
VIYADGSTNYNPSLKS

SEQ ID NO: 33 - protein, 12 aa, Mus musculus
ARAYGNYWYIDV

SEQ ID NO: 34 - protein, 11 aa, Artificial Sequence
  317-4B6 L-CDR1 or CDR-L1
KSSESVSNDVA

SEQ ID NO: 35 - protein, 7 aa, Mus musculus
YAFHRFT

SEQ ID NO: 36 - protein, 9 aa, Mus musculus
HQAYSSPYT

SEQ ID NO: 37 - protein, 10 aa, Mus musculus
GYTFTNYGMN

SEQ ID NO: 38 - protein, 17 aa, Artificial Sequence
  326-4A3 H-CDR2 or CDR-H2
WINNNNAEPTYAQDFRG

SEQ ID NO: 39 - protein, 7 aa, Mus musculus
ARDVMDY

SEQ ID NO: 40 - protein, 15 aa, Mus musculus
RASESVDNYGYSFMH

SEQ ID NO: 41 - protein, 7 aa, Mus musculus
RASNLES

SEQ ID NO: 42 - protein, 8 aa, Mus musculus
QQSKEYPT

SEQ ID NO: 43 - protein, 118 aa, Artificial Sequence
  317-4B2 pro-Vh
QVQLQESGPGLVKPSETLSLTCTVSGFSLTSYGVHWIRQPPGKGLEWIGVIYAGGSTNYNPSLKSRVTISKDTSKNQFSLKLSSVTAADTAVYYCARAYGNYWYIDVWGQGTTVTVSS

SEQ ID NO: 44 - protein, 107 aa, Artificial Sequence; identical to SEQ ID NO: 26
  317-4B2 pro-Vk

SEQ ID NO: 45 - protein, 118 aa, Artificial Sequence
  317-4B5 pro-Vh
QVQLQESGPGLVKPSETLSLTCTVSGFSLTSYGVHWIRQPPGKGLEWIGVIYAGGSTNYNPSLKSRVTISKDTSKNQVSLKLSSVTAADTAVYYCARAYGNYWYIDVWGQGTTVTVSS

SEQ ID NO: 46 - protein, 107 aa, Artificial Sequence; identical to SEQ ID NO: 26
  317-4B5 pro-Vk

SEQ ID NO: 47 - DNA, 354 bp, Artificial Sequence
  317-1 cDNA-Vh
caggtgcagctgcaggagtcgggaccaggactggtgaagccttcggagaccctgtccctcacctgcactgtctctgggttttcattaaccagctatggtgtacactggatccggcagcccccagggaagggactggagtggctgggggtcatatgggccggtggaagcacaaattataatccctccctcaagagtcgactgaccatatcaaaagacaactccaagagccaggtttccctgaagatgagctctgtgaccgctgcggacacggccgtgtattactgtgcgagagcctatggtaactactggtacatcgatgtctggggccaagggaccacggtcaccgtctcctca

SEQ ID NO: 48 - protein, 118 aa, Artificial Sequence
  317-1 pro-Vh
QVQLQESGPGLVKPSETLSLTCTVSGFSLTSYGVHWIRQPPGKGLEWLGVIWAGGSTNYNPSLKSRLTISKDNSKSQVSLKMSSVTAADTAVYYCARAYGNYWYIDVWGQGTTVTVSS

SEQ ID NO: 49 - DNA, 321 bp, Artificial Sequence
  317-1 cDNA-Vk
gacatcgtgatgacccagtctccagactccctggctgtgtctctgggcgagagggccaccatcaactgcaaggccagccagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggcggggggaccaagctggagatcaaa

SEQ ID NO: 50 - protein, 107 aa, Artificial Sequence
  317-1 pro-Vk
DIVMTQSPDSLAVSLGERATINCKASQSVSNDVAWYQQKPGQPPKLLINYAFHRFTGVPDRFSGSGYGTDFTLTISSLQAEDVAVYYCHQAYSSPYTFGGGTKLEIK

SEQ ID NO: 51 - protein, 114 aa, Artificial Sequence
  326-3B1 pro-Vh
QVQLVQSGSELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTLVTVSS

SEQ ID NO: 52 - protein, 110 aa, Artificial Sequence
  326-3B1 pro-Vk
DIVLTQSPASLAVSPGQRATITCRASESVDNYGYSFMHWYQQKPGQPPKLLIYRASNLESGVPARFSGSGSRTDFTLTINPVEANDTANYYCQQSKEYPTFGGGTKVEIK

SEQ ID NO: 53 - protein, 114 aa, Artificial Sequence
  326-3G1 pro-Vh
QVQLVQSGPELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTLVTVSS

SEQ ID NO: 54 - protein, 110 aa, Artificial Sequence; identical to SEQ ID NO: 52
  326-3G1 pro-Vk

SEQ ID NO: 55 - DNA, 342 bp, Artificial Sequence
  326-1 cDNA-Vh
caggtgcagctggtgcagagcggcagcgagctgaagaagcccggcgccagcgtgaaggtgagctgcaaggccagcggctacaccttcaccaactacggcatgaactgggtgagacaggcccccggccagggcctggagtggatgggctggatcaacaacaacaacggcgagcccacctacgcccagggcttcagaggcagattcgtgttcagcctggacaccagcgccagcaccgcctacctgcagatcagcagcctgaagaccgaggacaccgccgtgtacttctgcgccagagacgtgatggactactggggccagggcaccaccgtgaccgtgagcagc

SEQ ID NO: 56 - protein, 114 aa, Artificial Sequence
  326-1 pro-Vh
QVQLVQSGSELKKPGASVKVSCKASGYTFTNYGMNWVRQAPGQGLEWMGWINNNNGEPTYAQGFRGRFVFSLDTSASTAYLQISSLKTEDTAVYFCARDVMDYWGQGTTVTVSS

SEQ ID NO: 57 - DNA, 330 bp, Artificial Sequence
  326-1 cDNA-Vk
gacattgtgctgacccagtctccagcctccttggccgtgtctccaggacagagggccaccatcacctgcagagccagtgaaagtgttgataattatggctatagttttatgcactggtatcagcagaaaccaggacaacctcctaaactcctgatttaccgtgcatccaacctagaatctggggtcccagccaggttcagcggcagtgggtctaggaccgatttcaccctcacaattaatcctgtggaagctaatgatactgcaaattattactgtcagcaaagtaaagaatatccgacgttcggcggagggaccaaggtggagatcaaa

SEQ ID NO: 58 - protein, 110 aa, Artificial Sequence; identical to SEQ ID NO: 52
  326-1 pro-Vk

SEQ ID NO: 59 - protein, 16 aa, Artificial Sequence
  317-1 H-CDR2 or CDR-H2
VIWAGGSTNYNPSLKS

SEQ ID NO: 60 - protein, 16 aa, Artificial Sequence
  317-4B2 H-CDR2 or CDR-H2
VIYAGGSTNYNPSLKS

SEQ ID NO: 61 - protein, 11 aa, Artificial Sequence
  317-4B2 L-CDR1 or CDR-L1
KSSESVSNDVA

SEQ ID NO: 62 - protein, 17 aa, Artificial Sequence
  326-1 H-CDR2 or CDR-H2
WINNNNGEPTYAQGFRG

SEQ ID NO: 63 - protein, 17 aa, Artificial Sequence
  326-3G1 H-CDR2 or CDR-H2
WINNNNGEPTYAQDFRG

SEQ ID NO: 64 - protein, 118 aa, Artificial Sequence
  317-3A1 pro-Vh
QVQLQESGPGLVKPSETLSLTCTVSGFSLTSYGVHWIRQPPGKGLEWIGVIWAGGSTNYNPSLKSRVTISVDTSKNQFSLKLSSVTAADTAVYYCARAYGNYWYIDVWGQGTTVTVSS

SEQ ID NO: 65 - protein, 118 aa, Artificial Sequence
  317-3C1 pro-Vh
QVQLQESGPGLVKPSETLSLTCTVSGFSLTSYGVHWIRQPPGKGLEWLGVIWAGGSTNYNPSLKSRVTISVDTSKNQFSLKLSSVTAADTAVYYCARAYGNYWYIDVWGQGTTVTVSS

SEQ ID NO: 66 - protein, 118 aa, Artificial Sequence
  317-3E1 pro-Vh
QVQLQESGPGLVKPSETLSLTCTVSGFSLTSYGVHWIRQPPGKGLEWIGVIWAGGSTNYNPSLKSRVTISVDTSKSQFSLKLSSVTAADTAVYYCARAYGNYWYIDVWGQGTTVTVSS

SEQ ID NO: 67 - protein, 118 aa, Artificial Sequence
  317-3F1 pro-Vh
QVQLQESGPGLVKPSETLSLTCTVSGFSLTSYGVHWIRQPPGKGLEWIGVIWAGGSTNYNPSLKSRVTISVDTSKNQVSLKLSSVTAADTAVYYCARAYGNYWYIDVWGQGTTVTVSS

SEQ ID NO: 68 - protein, 118 aa, Artificial Sequence
  317-3G1 pro-Vh
QVQLQESGPGLVKPSETLSLTCTVSGFSLTSYGVHWIRQPPGKGLEWIGVIWAGGSTNYNPSLKSRVTISVDTSKSQVSLKLSSVTAADTAVYYCARAYGNYWYIDVWGQGTTVTVSS

SEQ ID NO: 69 - protein, 118 aa, Artificial Sequence
  317-3H1 pro-Vh
QVQLQESGPGLVKPSETLSLTCTVSGFSLTSYGVHWIRQPPGKGLEWIGVIWAGGSTNYNPSLKSRVTISKDTSKSQVSLKLSSVTAADTAVYYCARAYGNYWYIDVWGQGTTVTVSS

SEQ ID NO: 70 - protein, 118 aa, Artificial Sequence
  317-3I1 pro-Vh
QVQLQESGPGLVKPSETLSLTCTVSGFSLTSYGVHWIRQPPGKGLEWIGVIWAGGSTNYNPSLKSRVTISKDTSKNQVSLKLSSVTAADTAVYYCARAYGNYWYIDVWGQGTTVTVSS

SEQ ID NO: 71 - protein, 118 aa, Artificial Sequence
  317-4B1 pro-Vh
QVQLQESGPGLVKPSETLSLTCTVSGFSLTSYGVHWIRQPPGKGLEWIGVIWAGGSTNYNPSLKSRVTISKDTSKNQFSLKLSSVTAADTAVYYCARAYGNYWYIDVWGQGTTVTVSS

SEQ ID NO: 72 - protein, 118 aa, Artificial Sequence
  317-4B3 pro-Vh
QVQLQESGPGLVKPSETLSLTCTVSGFSLTSYGVHWIRQPPGKGLEWIGVINAGGSTNYNPSLKSRVTISKDTSKNQFSLKLSSVTAADTAVYYCARAYGNYWYIDVWGQGTTVTVSS

SEQ ID NO: 73 - protein, 118 aa, Artificial Sequence
  317-4B4 pro-Vh
QVQLQESGPGLVKPSETLSLTCTVSGFSLTSYGVHWIRQPPGKGLEWIGVIWAGGSTNYNPSLKSRVTISKDTSKNQFSLKLSSVTAADTAVYYCARAYGNYPYIDVWGQGTTVTVSS

SEQ ID NO: 74 - protein, 107 aa, Artificial Sequence
  317-4A2 pro-Vk
DIVMTQSPDSLAVSLGERATINCKSSQSVSNDVAWYQQKPGQPPKLLINYAFHRFTGVPDRFSGSGYGTDFTLTISSLQAEDVAVYYCHQAYSSPYTFGQGTKLEIK

SEQ ID NO: 75 - protein, 114 aa, Artificial Sequence
  326-3A1 pro-Vh
QVQLVQSGPELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTLVTVSS

SEQ ID NO: 76 - protein, 114 aa, Artificial Sequence
  326-3C1 pro-Vh
QVQLVQSGPELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTTVTVSS

SEQ ID NO: 77 - protein, 114 aa, Artificial Sequence
  326-3D1 pro-Vh
QVQLVQSGSELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTLVTVSS

SEQ ID NO: 78 - protein, 114 aa, Artificial Sequence
  326-3E1 pro-Vh
QVQLVQSGSELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTTVTVSS

SEQ ID NO: 79 - protein, 114 aa, Artificial Sequence
  326-3F1 pro-Vh
QVQLVQSGPELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTTVTVSS

SEQ ID NO: 80 - protein, 114 aa, Artificial Sequence
  326-3B N55D pro-Vh
QVQLVQSGSELKKPGASVKVSCKASGYTFTNYGMNWVRQAPGQGLKWMGWINNNDGEPTYAQDFRGRFVFSLDTSASTAYLQISSLKTEDTAVYYCARDVMDYWGQGTLVTVSS

SEQ ID NO: 81 - protein, 110 aa, Artificial Sequence
  326-4A1 pro-Vk
DIVLTQSPASLAVSPGQRATITCRASESVDNYGYSFMHWYQQKPGQPPKLLIYRASNLESGVPARFSGSGSRTDFTLTINPVEAEDTANYYCQQSKEYPTFGGGTKVEIK

SEQ ID NO: 82 - protein, 110 aa, Artificial Sequence
  326-4A2 pro-Vk
DIVLTQSPASLAVSPGQRATITCRASESVDNYGYSFMHWYQQKPGQPPKLLIYRASNLESGVPARFSGSGSGTDFTLTINPVEANDTANYYCQQSKEYPTFGGGTKVEIK

SEQ ID NO: 83 - protein, 327 aa, Artificial Sequence
  huIgG4mt1 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 327 aa, Artificial Sequence
  huIgG4mt2 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 327 aa, Artificial Sequence
  huIgG4mt6 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 327 aa, Artificial Sequence
  huIgG4mt8 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVTVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 327 aa, Artificial Sequence
  huIgG4mt9 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 88 - protein, 327 aa, Artificial Sequence
  huIgG4mt10 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 89 - protein, 367 aa, Artificial Sequence
  OS8 pro
MERHWIFLLLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 90 - protein, 304 aa, Artificial Sequence
  P3Z pro
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2022/0387404 A1
(43) Pub. Date: Dec. 8, 2022

(54) TREATMENT OF CANCER USING A COMBINATION COMPRISING MULTI-TYROSINE KINASE INHIBITOR AND IMMUNE CHECKPOINT INHIBITOR

(71) Applicant: BEIGENE, LTD., Camana Bay, Grand Cayman (KY)

(72) Inventors: Beibei JIANG, Beijing (CN); Liu YANG, Beijing (CN); Cheng CHEN, Beijing (CN)

(21) Appl. No.: 17/642,125

(22) PCT Filed: Sep. 11, 2020
(86) PCT No.: PCT/CN2020/114703

§ 371 Date: Mar. 10, 2022

(30) Foreign Application Priority Data
Sep. 11, 2019 (CN) PCT/CN2019/105418

(51) Int. Cl. A61K 31/444; C07K 16/28; A61P 35/04

(52) U.S. Cl. CPC A61K 31/444; C07K 16/2818; A61P 35/04; C07B 2200/13

(57) ABSTRACT

Provided herein is a method for the prevention, delay of progression or treatment of cancer in a subject, comprising administering to the subject in need thereof a multi-tyrosine kinase inhibitor, N-(3-fluoro-4-((2-(5-(((2-methoxyethyl)amino)methyl)pyridin-2-yl)thieno[3, 2-b]pyridin-7-yl)oxy)phenyl)-N-(4-fluorophenyl)cyclopropane-1, 1-dicarboxamide or a pharmaceutically acceptable salt thereof, in combination with an immune checkpoint inhibitor. Also, provided a pharmaceutical combination comprising a multi-tyrosine kinase inhibitor, N-(3-fluoro-4-((2-(5-(((2-methoxyethyl)amino)methyl)pyridin-2-yl)thieno[3, 2-b]pyridin-7-yl)oxy)phenyl)-N-(4-fluorophenyl)cyclopropane-1, 1-dicarboxamide or a pharmaceutically acceptable salt thereof, in combination C with an immune checkpoint inhibitor and the use thereof.

### CROSS REFERENCE OF RELATED APPLICATIONS

This application claims the benefit of priority of International Patent Application No. PCT/CN2019/105418 filed on Sep. 11, 2019, the disclosure of which is hereby incorporated by reference in its entirety for all purposes.

### DESCRIPTION OF THE TEXT FILE SUBMITTED ELECTRONICALLY

The contents of the text file submitted electronically herewith are incorporated herein by reference in its entirety: A computer-readable format copy of the Sequence Listing (file name: L10205-01PCT_SeqList, data recorded Sep. 11, 2020)

### FIELD OF THE DISCLOSURE

Disclosed herein is a method for the prevention, delay of progression or treatment of cancer in a subject, comprising administering to the subject in need thereof a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a pharmaceutically acceptable salt thereof) in combination with an immune checkpoint inhibitor. Disclosed herein is also a pharmaceutical combination comprising a multi-tyrosine kinase inhibitor (for example, N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a pharmaceutically acceptable salt thereof) in combination with an immune checkpoint inhibitor and the use thereof.

### BACKGROUND OF THE DISCLOSURE

Cancer cells face selective pressures while being treated and mutations occurring in individual cancer cells represent the continuous evolution of original cancer. Many malignancies develop resistance to individual anticancer therapies. This is also the case for immune checkpoint blockade agents where acquired resistance occurs in a large portion of treated patients who achieved an initial meaningful response. This phenomenon of acquired resistance helps cancer cells adapt to the environment and survive immune attacks and is a reminder of therapeutic challenges that need to be overcome [Syn N L, Teng M W L, Mok T S K, et al. De-novo and acquired resistance to immune checkpoint targeting. Lancet Oncol. 2017;18(12): e731-41.].

Monoclonal antibodies that target either PD-1 or PD-L1, can block this interaction and boost the immune response against cancer cells. These antibodies have been shown to be helpful in treating several types of cancer, including melanoma of the skin, non-small cell lung cancer (NSCLC), kidney cancer, bladder cancer, head and neck cancers, and Hodgkin lymphoma. Cancer cells in most non-responders to single-agent checkpoint inhibitors escape through innate mechanisms that allow the cancer cells to grow and survive. As a result, the disease progresses at a rate consistent with the natural history. However, unlike intrinsic resistance, late relapses are now emerging in patients with prior clinical benefit after longer follow-up of clinical trials, suggesting the emergence of acquired resistance [Jenkins R W, Barbie D A, and Flaherty K T. Mechanisms of resistance to immune checkpoint inhibitors. Br J Cancer. 2018; 118(1):9-16.]. Strategies to improve the clinical efficacy of checkpoint inhibitors by overcoming innate or acquired resistance are needed.

### SUMMARY OF THE DISCLOSURE

International publication No. WO2009/026717A disclosed compounds with the inhibition activities of multiple protein tyrosine kinases, for example, the inhibition activities of VEGF receptor kinase and HGF receptor kinase. In particular, disclosed herein is N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide (Compound 1) which is a multi-tyrosine kinase inhibitor with demonstrated potent inhibition of a closely related spectrum of tyrosine kinases, including RET, CBL, CHR4q12, DDR and Trk, which are key regulators of signaling pathways that lead to cell growth, survival and tumor progression.

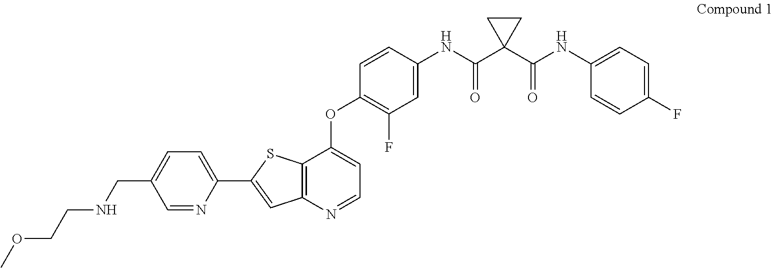 [SMILES: COCCNCc1ccc(-c2cc3nccc(Oc4ccc(NC(=O)C5(C(=O)Nc6ccc(F)cc6)CC5)cc4F)c3s2)nc1]

The inventors of the present application have found that the combination of a Multi-tyrosine kinase inhibitor (for example, the above-mentioned Compound 1) with an immune checkpoint inhibitor (for example, anti-PD-1 antibody Mab-1 in the present disclosure) produces significant inhibition of tumor growth in cancers as compared with the monotherapy of each of the above active pharmaceutical agent alone. The combination treatment with Mab-1 and Compound 1 is promising, with antitumor activity in patients with a variety of cancers including ovarian cancer.

In a first aspect, disclosed herein is a method for the prevention, delay of progression or treatment of cancer in a subject, comprising administering to the subject in need thereof a therapeutically effective amount of a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a stereoisomer thereof, or a pharmaceutically acceptable salt thereof), in combination with a therapeutically effective amount of an immune checkpoint inhibitor.

In a second aspect, disclosed herein is a pharmaceutical combination for use in the prevention, delay of progression or treatment of cancer, comprising a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a stereoisomer thereof, or a pharmaceutically acceptable salt thereof), in combination with an immune checkpoint inhibitor.

In a third aspect, disclosed herein is a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a stereoisomer thereof, or a pharmaceutically acceptable salt thereof), for use in the prevention, delay of progression or treatment of cancer in combination with an immune checkpoint inhibitor. In one embodiment of this aspect, disclosed herein is an immune checkpoint inhibitor for use in the prevention, delay of progression or treatment of cancer in combination with a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a stereoisomer thereof, or a pharmaceutically acceptable salt thereof).

In a fourth aspect, disclosed herein is a use of a pharmaceutical combination in the manufacture of a medicament for use in the prevention, delay of progression or treatment of cancer, said pharmaceutical combination comprising a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a stereoisomer thereof, or a pharmaceutically acceptable salt thereof), and an immune checkpoint inhibitor.

In a fifth aspect, disclosed herein is an article of manufacture, or “kit” comprising a first container, a second container and a package insert, wherein the first container comprises at least one dose of a medicament comprising a multi-tyrosine kinase inhibitor (for example N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a stereoisomer thereof, or a pharmaceutically acceptable salt thereof), the second container comprises at least one dose of a medicament comprising an immune checkpoint inhibitor, and the package insert comprises instructions for treating cancer a subject using the medicaments.

The method and pharmaceutical combination disclosed herein, as a combination therapy, are significantly more efficacious than administration of one of the therapeutics.

In an embodiment of each of the above five aspects, the immune checkpoint inhibitor is a monoclonal antibody. In an embodiment of each of the above five aspects, the immune checkpoint inhibitor is an anti-PD-1 antibody.

In an embodiment of each of the above five aspects, the cancer is selected from lung cancer including non-small cell lung cancer (NSCLC), ovarian cancer (OC), renal cell carcinoma (RCC) or melanoma.

In an embodiment of each of the above five aspects, the cancer or tumor is resistant or refractory to a checkpoint inhibitor selected from an anti-PD-1 antibody or anti-PD-L1 antibody.

In an embodiment of present disclosure, the cancer is PD-L1 expression.

In an embodiment of present disclosure, the cancer is selected by its stage including locally advanced, recurrent or metastatic.

In an embodiment of present disclosure, the cancer is non-squamous non-small cell lung cancer (NSCLC), squamous non-small cell lung cancer (NSCLC), epithelial ovarian cancer (OC), renal cell carcinoma (RCC) or melanoma.

In an embodiment of present disclosure, the non-squamous non-small cell lung cancer (NSCLC) is: anti-PD-1/PD-L1 antibody refractory/resistant metastatic, non-squamous NSCLC; anti-PD-1/PD-L1 antibody naïve metastatic, non-squamous NSCLC; or, PD-L1 positive, locally advanced or metastatic, non-squamous NSCLC without prior systemic treatment in the metastatic setting.

In an embodiment of present disclosure, the squamous non-small cell lung cancer (NSCLC) is: anti-PD-1/PD-L1 antibody refractory/resistant metastatic, squamous NSCLC; anti-PD-1/PD-L1 antibody naïve metastatic, squamous NSCLC; or, PD-L1 positive, locally advanced or metastatic, squamous NSCLC without prior systemic treatment in the metastatic setting.

In an embodiment of present disclosure, the renal cell carcinoma (RCC) is anti-PD-1/PD-L1 antibody refractory/resistant metastatic or advanced RCC, or metastatic or advanced RCC without prior systemic therapy.

In an embodiment of present disclosure, the melanoma is anti-PD-1/PD-L1 antibody refractory/resistant unresectable or metastatic melanoma.

In one embodiment of present disclosure, the ovarian cancer (OC) is naïve recurrent and platinum-resistant epithelial OC.

In an embodiment of each of the above five aspects, the immune checkpoint inhibitor is a PD-1 antagonist, which is an antibody (Mab-1) comprising a heavy chain variable region (Vh) and a light chain variable region (Vk), and an IgG4 heavy chain effector or constant domain comprising SEQ ID NO: 88, wherein the heavy chain variable region (Vh) and the light chain variable region (Vk) comprises SEQ ID NO: 24 and SEQ ID NO: 26, respectively.

In an embodiment of each of the above five aspects, the multi-tyrosine kinase inhibitor is N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide (Compound 1). In one embodiment, Compound 1 is in Crystalline Form D. In another embodiment, the Crystalline Form D has an XRPD pattern substantially as shown in FIG. 1A.

In an embodiment of each of the above five aspects, the multi-tyrosine kinase inhibitor and the immune checkpoint inhibitor, are administered simultaneously, sequentially or intermittently.

### BRIEF DESCRIPTION OF THE DRAWINGS

FIG. 1A illustrates an X-ray powder diffraction (XRPD) pattern of Crystalline Form D of Compound 1 (Compound 1 Form D).

FIG. 2A shows the combination efficacy of Mab-1 and Compound 1 in an A431 allogeneic mouse model (* indicates P<0.05).

FIG. 2B shows the combination efficacy of muCh15mt and Compound 1 in a CT26WT syngeneic mouse model (*** indicates P<0.001).

FIG. 2C shows the combination efficacy of muCh15mt and Compound 1 in an MC38 syngeneic mouse model (**** indicates P<0.0001).

FIG. 2D shows the combination efficacy of muCh15mt and Compound in an A20 syngeneic mouse model (* indicates P<0.05).

### DETAILED DESCRIPTION

### Definitions

Unless specifically defined elsewhere in this document, all other technical and scientific terms used herein have the meaning commonly understood by one of ordinary skill in the art to which this disclosure belongs.

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

Throughout this specification and the claims which follow, unless the context requires otherwise, the word “comprise”, and variations such as “comprises” and “comprising”, will be understood to imply the inclusion of an active agent (e.g., a mAb or a multi-tyrosine kinase inhibitor) or a stated amino acid sequence, but not the exclusion of any other active ingredient or amino acid sequence.

The term “Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. It includes, but are not limited to salts with inorganic acids, selected, for example, from hydrochlorates, phosphates, diphosphates, hydrobromates, sulfates, sulfinates, and nitrates; as well as salts with organic acids, selected, for example, from fumarates, lactates, methanesulfonates, p-toluenesulfonates, 2-hydroxyethylsulfonates, benzoates, salicylates, stearates, alkanoates such as acetate, and salts with HOOC—(CH₂)ₙ—COOH, wherein n is selected from 0 to 4. Similarly, examples of pharmaceutically acceptable cations include, but are not limited to, sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of Compound 1, and salts of the stereoisomers of the Compound 1, such as salts of enantiomers, and/or salts of diastereomers.

An “effective amount” refers to an amount of at least one Compound 1 and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent the development of, or alleviate to some extent, one or more of the symptoms of the condition or disorder being treated. The therapeutically effective amount will vary depending on the compound, the disease and its severity and the age, weight, etc., of the mammal to be treated.

The term “at least one substituent” includes, for example, from 1 to 4, such as from 1 to 3, further as 1 or 2, substituents. For example, “at least one substituent R¹⁶” herein includes from 1 to 4, such as from 1 to 3, further as 1 or 2, substituents selected from the list of R<16>as described herein.

The term “antibody” herein is used in the broadest sense and specifically covers antibodies (including full length monoclonal antibodies) and antibody fragments so long as they recognize antigen, such as, an immune checkpoint (e.g., PD-1). An antibody molecule is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. Monoclonal antibodies (mAbs) may be obtained by methods known to those skilled in the art. See, for example, U.S. Pat. No. 4,376,110. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained in in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (Vl/Vh) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs)”, which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5<m> ed.; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al, (1989) Nature 342:878-883.

The term “hypervariable region” means the amino acid residues of an antibody that are responsible for antigen-binding. The hypervariable region comprises amino acid residues from a “complementarity determining region” or “CDR” (i.e., CDR-L1, CDR-L2 and CDR-L3 in the light chain variable domain and CDR-H1, CDR-H2 and CDR-H3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” means those variable domain residues other than the hypervariable region residues defined herein as CDR residues.

Unless otherwise indicated, “antibody fragment” or “antigen-binding fragment” means antigen binding fragments of antibodies, i.e. antibody fragments that retain the ability to bind specifically to the antigen bound by the full-length antibody, e.g. fragments that retain one or more CDR regions. Examples of antigen binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies or binding fragments thereof, useful in the present disclosure will bind to the target protein with an affinity that is at least two fold greater, preferably at least ten times greater, more preferably at least 20-times greater, and most preferably at least 100-times greater than the affinity with non-target proteins. An antibody herein is said to bind specifically to a polypeptide comprising a given amino acid sequence, e.g. the amino acid sequence of a mature human PD-1 molecule, if it binds to polypeptides comprising that sequence but does not bind to proteins lacking that sequence.

The term “human antibody” herein means an antibody that comprises human immunoglobulin protein sequences only. A human antibody may contain rodent carbohydrate chains if produced in a mouse, in a rodent cell, or in a hybridoma derived from a mouse cell. Similarly, “mouse antibody” or “rat antibody” means an antibody that comprises only mouse or rat immunoglobulin protein sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu”, “Hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase the stability of the humanized antibody, or for other reasons.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302]. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

The terms “administration,” “administering,” “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “treating” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The phrase “reducing the likelihood” refers to delaying the onset or development or progression of the disease, infection or disorder.

The term “therapeutically acceptable amount” or “therapeutically effective dose” interchangeably refers to an amount sufficient to affect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of, or result in a decrease in the severity of, respectively, disease symptoms, including symptoms associated polyoma viral infection.

The term “co-administer” refers to the simultaneous presence of two active agents in the blood of an individual. Active agents that are co-administered can be concurrently or sequentially delivered.

An “effective amount” refers to an amount of at least one Compound Ind/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent the development of, or alleviate to some extent, one or more of the symptoms of the condition or disorder being treated. The therapeutically effective amount will vary depending on the compound, the disease and its severity and the age, weight, etc., of the mammal to be treated.

The terms “cancer” or “tumor” herein mean or describe the physiological condition involving abnormal cell growth with the potential to invade or spread to other parts of the body.

As used herein, the term “resistant,” “resistant cancer” or “refractory” refers to a condition wherein the cancer demonstrates reduced sensitivity to a therapeutic. For example, in a resistant cancer, fewer cancer cells are eliminated by the concentration of a therapeutic that is used to eliminate cancer cells in a sensitive cancer of the same type. A cancer can be resistant at the beginning of a therapeutic treatment or it can become resistant during treatment. Resistance can be due to several mechanisms such as but not limited to; alterations in drug-targets, decreased drug accumulation, alteration of intracellular drug distribution, reduced drug-target interaction, increased detoxification response, cell-cycle deregulation, increased damaged-DNA repair, and reduced apoptotic response. Several of said mechanisms can occur simultaneously and/or can interact with each other.

The term “disease” refers to any disease, discomfort, illness, symptoms or indications, and can be substituted with the term “disorder” or “condition.”

The term “pharmaceutical combination” as used herein refers to either a fixed combination in one dosage unit form, or non-fixed combination or a kit of parts for the combined administration where two or more therapeutic agents can be administered independently at the same time or separately within time intervals, especially where these time intervals allow that the combination partners show a cooperative, e.g., synergistic effect.

The term “combination therapy” or “combination treatment” refers to the administration of two or more therapeutic agents to treat cancer or a consequence of cancer as described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids can be reconstituted or diluted to the desired dose prior to administration. In addition, such administration also encompasses the use of each type of therapeutic agent in a sequential manner, either at approximately the same time or at different times. In either case, the treatment regimen will provide beneficial effects of the therapeutic combination in treating the conditions or disorders described herein.

Unless specifically defined elsewhere in this document, all other technical and scientific terms used herein have the meaning commonly understood by one of ordinary skill in the art to which this disclosure belongs.

The combination therapy can provide “synergy” and prove “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In some embodiments of the present disclosure, the multi-tyrosine kinase inhibitor is co-administered with a PD-1 antagonist. In some embodiments of the present disclosure, the PD-1 antagonist is an antibody or a fragment antigen binding thereof.

PD-1 Antagonist

PD-1 is an immune checkpoint protein, that limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD-1 blockade can be accomplished by a variety of mechanisms including antibodies that bind PD-1 or its ligands.

In some embodiments of the present disclosure, the immune checkpoint inhibitor is a PD-1 antagonist, which is a monoclonal antibody or a fragment thereof, disclosed in WO 2015/035606 A1. In some embodiments of the present disclosure, the immune checkpoint inhibitor is an anti-PD-1 antibody.

Preferably, the anti-PD-1 monoclonal antibody is an antibody which comprises a heavy chain variable region (Vh) and a light chain variable region (Vl) that contain complement determinant regions (CDRs) listed as follows in Table 1.

TABLE 1

| | |
|---|---|
| a) mu317 | CDR-H1, CDR-H2 and CDR-H3 (SEQ ID NOs: 11, 12, 13, respectively); and |
| | CDR-L1, CDR-L2 and CDR-L3 (SEQ ID NOs: 14, 15, 16, respectively); |
| b) mu326 | CDR-H1, CDR-H2 and CDR-H3 (SEQ ID NOs: 17, 18, 19, respectively); and |
| | CDR-L1, CDR-L2 and CDR-L3 (SEQ ID NOs: 20, 21, 22, respectively); |
| c) 317-4B6 | CDR-H1, CDR-H2 and CDR-H3 (SEQ ID NOs: 31, 32, 33, respectively); and |
| | CDR-L1 , CDR-L2 and CDR-L3 (SEQ ID NOs: 34, 35, 36, respectively); |
| d) 326-4A3 | CDR-H1, CDR-H2 and CDR-H3 (SEQ ID NOs: 37, 38, 39, respectively); and |
| | CDR-L1, CDR-L2 and CDR-L3 (SEQ ID NOs: 40, 41, 42, respectively); |
| e) 317-1H | CDR-H1, CDR-H2 and CDR-H3 (SEQ ID NOs: 11, 59, 13, respectively); and |
| | CDR-L1, CDR-L2 and CDR-L3 (SEQ ID NOs: 14, 15, 16, respectively); |
| f) 317-4B2 | CDR-HL CDR-H2 and CDR-H3 (SEQ ID NOs: 11, 60, 13, respectively); and |
| | CDR-L1, CDR-L2 and CDR-L3 (SEQ ID NOs: 61 , 15, 16, respectively); |
| g) 317-4B5 | CDR-H1, CDR-H2 and CDR-H3 (SEQ ID NOs: 11 , 60, 13, respectively); and |
| | CDR-L1, CDR-L2 and CDR-L3 (SEQ ID NOs: 61 , 15, 16, respectively); |
| h) 317-4B6 | CDR-H1, CDR-H2 and CDR-H3 (SEQ ID NOs: 11, 32, 13, respectively); and |
| | CDR-L1, CDR-L2 and CDR-L3 (SEQ ID NOs: 61, 15, 16, respectively); |
| i) 326-1 | CDR-H1, CDR-H2 and CDR-H3 (SEQ ID NOs: 17, 62, 19, respectively); and |
| | CDR-L1, CDR-L2 and CDR-L3 (SEQ ID NOs: 20, 21, 22, respectively); |
| j) 326-3B1 | CDR-H1, CDR-H2 and CDR-H3 (SEQ ID NOs: 17, 62, 19, respectively); and |
| | CDR-L1, CDR-L2 and CDR-L3 (SEQ ID NOs: 20, 21, 22, respectively); |
| or k) 326-3G1 | CDR-H1, CDR-H2 and CDR-H3 (SEQ ID NOs: 17, 62, 19, respectively); and |
| | CDR-L1, CDR-I2 and CDR-L3 (SEQ ID NOs: 20, 21, 22, respectively). |

Preferably, the anti-PD-1 monoclonal antibody is an antibody which comprises a heavy chain variable region (Vh) and a light chain variable region (Vl) that contain any combinations of CDRs listed as follows in Table 2.

TABLE 2

| | |
|---|---|
| (a) | CDR-H1 (SEQ ID NO 31), CDR-H2 |
| | (SEQ ID NO 12, 32, 5 9 or 60) |
| | and CDR-H3 (SEQ ID NO 33), |
| | CDR-L1 (SEQ ID NO 14, 34 or 61), |
| | CDR-L2 (SEQ ID NO 35) and CDR-L3 |
| | (SEQ ID NO 36); or |
| (b) | CDR-H1 (SEQ ID NO 37), CDR-H2 |
| | (SEQ ID NO 18, 38 or 62) |
| | and CDR-H3 (SEQ ID NO 39), CDR-L1, |
| | (SEQ ID NO 40) CDR-L2 (SEQ ID NO 41) |
| | and CDR-L3 (SEQ ID NO 42). |

Preferably, the anti-PD-1 monoclonal antibody is an antibody which comprises a heavy chain variable region (Vh) and a light chain variable region (V) listed as follows in

TABLE 3
a) mu317 (SEQ ID NOs: 4 and 6, respectively);
b) mu326 (SEQ ID NOs: 8 and 10, respectively);
c) 317-4B6 (SEQ ID NOs: 24 and 26, respectively);
d) 326-4A3 (SEQ ID NOs: 28 and 30, respectively);
e) 317-4B2 (SEQ ID NOs: 43 and 44, respectively);
f) 317-4B5 (SEQ ID NOs: 45 and 46, respectively);
g) 317-1 (SEQ ID NOs: 48 and 50, respectively);
h) 326-3B1 (SEQ ID NOs: 51 and 52, respectively);
i) 326-3GI (SEQ ID NOs: 53 and 54, respectively);
j) 326-1 (SEQ ID NOs: 56 and 58, respectively);
k) 317-3A1 (SEQ ID NOs: 64 and 26, respectively);
l) 317-3C1 (SEQ ID NOs: 65 and 26, respectively);
m) 317-3E1 (SEQ ID NOs: 66 and 26, respectively);
n) 317-3F1 (SEQ ID NOs: 67 and 26, respectively);
o) 317-3G1 (SEQ ID NOs: 68 and 26, respectively);
p) 317-3H1 (SEQ ID NOs: 69 and 26, respectively);
q) 317-311 (SEQ ID NOs: 70 and 26, respectively);
r) 317-4B1 (SEQ ID NOs: 71 and 26, respectively);
s) 317-4B3 (SEQ ID NOs: 72 and 26, respectively);
t) 317-4B4 (SEQ ID NOs: 73 and 26, respectively);
n) 317-4A2 (SEQ ID NOs: 74 and 26, respectively);
v) 326-3A1 (SEQ ID NOs: 75 and 30, respectively);
w) 326-3C1 (SEQ ID NOs: 76 and 30, respectively);
x) 326-3D1 (SEQ ID NOs: 77 and 30, respectively);
y) 326-3E1 (SEQ ID NOs: 78 and 30, respectively);
z) 326-3F1 (SEQ ID NOs: 79 and 30, respectively);
aa) 326-3B N55D (SEQ ID NOs: 80 and 30, respectively);
ab) 326-4A1 (SEQ ID NOs: 28 and 81, respectively); or
ac) 326-4A2 (SEQ ID NOs: 28 and 82, respectively).

Preferably, the anti-PD-1 monoclonal antibody is an antibody which comprises an IgG4 heavy chain effector or constant domain comprising any of SEQ ID NOs: 83-88.

Preferably, the anti-PD-1 monoclonal antibody is an antibody which contains an F(ab) or F(ab)2 comprising a domain said above, including a heavy chain variable region (Vh), a light chain variable region (VI), and an IgG4 heavy chain effector or constant domain.

Preferably, the anti-PD-1 monoclonal antibody is an antibody which comprises a heavy chain variable region (Vh) and a light chain variable region (VI), and an IgG4 heavy chain effector or constant domain comprising SEQ ID NOs: 87 or 88, wherein the heavy chain variable region (Vh) and the light chain variable region (Vl) are in Table 4.

TABLE 4
a) mu317 (SEQ ID NOs: 4 and 6, respectively);
b) mu326 (SEQ ID NOs: 8 and 10, respectively);
c) 317-4B6 (SEQ ID NOs: 24 and 26, respectively);
d) 326-4A3 (SEQ ID NOs: 28 and 30, respectively);
e) 317-4B2 (SEQ ID NOs: 43 and 44, respectively);
f) 317-4B5 (SEQ ID NOs: 45 and 46, respectively);
g) 317-1 (SEQ ID NOs: 48 and 50, respectively);
h) 326-3B1 (SEQ ID NOs: 51 and 52, respectively);
i) 326-3GI (SEQ ID NOs: 53 and 54, respectively);
j) 326-1 (SEQ ID NOs: 56 and 58, respectively);
k) 317-3A1 (SEQ ID NOs: 64 and 26, respectively);
l) 317-3C1 (SEQ ID NOs: 65 and 26, respectively);
m) 317-3E1 (SEQ ID NOs: 66 and 26, respectively);
n) 317-3F1 (SEQ ID NOs: 67 and 26, respectively);
o) 317-3G1 (SEQ ID NOs: 68 and 26, respectively);
p) 317-3H1 (SEQ ID NOs: 69 and 26, respectively);
q) 317-311 (SEQ ID NOs: 70 and 26, respectively);
r) 317-4B1 (SEQ ID NOs: 71 and 26, respectively);
s) 317-4B3 (SEQ ID NOs: 72 and 26, respectively);
t) 317-4B4 (SEQ ID NOs: 73 and 26, respectively);
u) 317-4A2 (SEQ ID NOs: 74 and 26, respectively);
v) 326-3A1 (SEQ ID NOs: 75 and 30, respectively);
w) 326-3C1 (SEQ ID NOs: 76 and 30, respectively);
x) 326-3D1 (SEQ ID NOs: 77 and 30, respectively);
y) 326-3E1 (SEQ ID NOs: 78 and 30, respectively);
z) 326-3F1 (SEQ ID NOs: 79 and 30, respectively);
aa) 326-3B N55D (SEQ ID NOs: 80 and 30, respectively);
ab) 326-4A1 (SEQ ID NOs: 28 and 81, respectively); or
ac) 326-4A2 (SEQ ID NOs: 28 and 82, respectively).

Preferably, the anti-PD-1 monoclonal antibody is an antibody which comprises a heavy chain variable region (Vh) and a light chain variable region (Vl) (comprising SEQ ID No 24 and SEQ ID No 26, respectively) and an IgG4 heavy chain effector or constant domain (comprising SEQ ID NO 88), hereinafter Mab-1, which specifically binds to PD-1, especially PD-1 residues including K45 and 193; or, 193, L95, and P97, and inhibits PD-1-mediated cellular signaling and activities in immune cells, antibodies binding to a set of amino acid residues required for its ligand binding.

The anti-PD1 monoclonal antibody and antibody fragment thereof may be prepared in accordance with the disclosure of WO2015/035606 A1, the entire disclosure of which is expressly incorporated herein by reference. In one embodiment of the present disclosure, the anti-PD1 monoclonal antibody is Mab-1, which is administered parenterally at a dosage of about a dose of 30-300 mg QW, or Q2W, or Q3W, or Q4W. In a preferred embodiment of present disclosure, the anti-PD1 monoclonal antibody is Mab-1, which is administered parenterally at a dosage of about at a dose of 200 mg Q3W.

### Combination Therapy

The combination therapy may be administered as a simultaneous or separate or sequential regimen. When administered sequentially, the combination may be administered in two or more administrations. The combined administration includes co-administration, using a separate formulation, and consecutive administration in either order, wherein preferably there is a time period while both (or all) active agents simultaneously exert their biological activities.

Suitable dosages for any of the above co-administered agents are those presently used and may be lowered due to the combined action (synergy) of the multi-tyrosine kinase inhibitor and the anti-PD-1 antibody, such as to increase the therapeutic index or mitigate toxicity or other side-effects or consequences.

In a particular embodiment of anti-cancer therapy, the multi-tyrosine kinase inhibitor and the anti-PD-1 antibody may be further combined with surgical therapy and radiotherapy.

In an embodiment of each of the above five aspects, the amounts of the multi-tyrosine kinase inhibitor and the anti-PD-1 antibody disclosed herein and the relative timings of administration are to be determined by the individual needs of the patient to be treated, administration route, the severity of disease or illness, dosing schedule, as well as evaluation and judgment of the designated doctor.

The multi-tyrosine kinase inhibitor and the anti-PD-1 antibody disclosed herein can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

In one embodiment of each of the above five aspects, the multi-tyrosine kinase inhibitor and the anti-PD-1 antibody disclosed herein may be administered in a different route. In a preferred embodiment, the multi-tyrosine kinase inhibitor is administered orally, and the anti-PD-1 antibody is administered parenterally such as subcutaneously, intracutaneously, intravenously or intraperitoneally.

In a preferred embodiment, the multi-tyrosine kinase inhibitor (in particularly N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide or a pharmaceutically acceptable salt thereof) is administered once a day (once daily, QD), two times per day (twice daily, BID), three times per day, four times per day, or five times per day, and is administered at a dosage of about 40 mg/day to about 640 mg/day. In a preferred embodiment, the multi-tyrosine kinase inhibitor is administered at a dose of from 40 mg QD to 200 QD. In a preferred embodiment, the multi-tyrosine kinase inhibitor is administered at a dose of from 40 mg QD to 150 mg QD. In a more preferred embodiment, the multi-tyrosine kinase inhibitor is administered at a dose of from 60 mg QD to 150 mg QD. In a most preferred embodiment, the multi-tyrosine kinase inhibitor is administered at a dose of 120 mg QD.

In a preferred embodiment, the anti-PD-1 antibody (in particularly an anti-PD1 monoclonal antibody Mab-1) is administered at a dose of 200 mg IV once every 3 weeks (Q3W).

### EXAMPLE

The present disclosure is further exemplified, but not limited to, by the following examples that are illustrated herein.

Example 1 illustrates the preparation of Crystalline Form D of N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide (Compound 1 Form D). And, Compound 1 Form D were further used in preclinical and clinical trial studies herein.

### Example 1

### Preparation of Crystalline Form D of N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide (Compound 1 Form D)

### Example 1A: Preparation of Compound 1

### Step 1: N-((6-bromopyridin-3-yl)methyl)-2-methoxyethan-1-amine (Compound 1A)

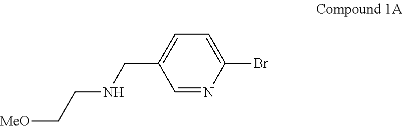 [SMILES: COCCNCc1ccc(Br)nc1]

To a stirred solution of 2-Methoxyethylamine (3.0 eq) in dichloromethane (DCM) (12 vol) was added Molecular sieves (0.3 w/w) and stirred for 2 hours at 25±5° C. under nitrogen atmosphere. The reaction mass water content was monitored by Karl Fischer analysis until the water content limit reached 0.5% w/w. Once the water content limit was reached, the reaction mass cooled to 5±5° C. and 6-bromonicotinaldehyde (1.0 eq) were added in a lot wise over 30 minutes to the above reaction mass at 5±5° C. The reaction mass was stirred for 30±5 minutes at 5±5° C. and acetic acid (1.05 eq) was added drop wise at 5±5° C. After completion of the addition, the mass was slowly warmed to 25±5° C. and stirred for 8 h to afford Compound 1A. The imine formation was monitored by HPLC.

### Step 2: tert-butyl ((6-bromopyridin-3-yl)methyl)(2-methoxyethyl)carbamate (Compound 1B)

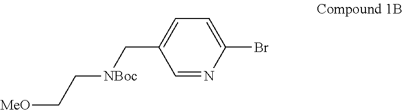 [SMILES: COCCN(Cc1ccc(Br)nc1)C(=O)OC(C)(C)C]

Charged Compound 1A (1.0 eq) in THE (5.0 vol) was added and the reaction mass was stirred for 30 minutes at 25±5° C. under nitrogen atmosphere. The reaction mass was cooled to the temperature of about 10±5° C. Di-tert-butyl dicarbonate (1.2 eq) was added to the reaction mass at 10±5° C. under nitrogen atmosphere and the reaction mass temperature was raised to 25±5° C. and the reaction mass for about 2 hours. The progress of the reaction was monitored by HPLC. After IPC completion, a prepared solution of Taurine (1.5 eq) in 2M aq NaOH (3.1 vol) was charged and stirred at 10±5° C. for 16 h to 18 h. The reaction mass was further diluted with 1 M aq NaOH solution (3.7 vol) and the layers were separated. The aqueous layer was extracted with DCM (2×4.7vol) and the extract combined with the organic layer. The combined organic layers were washed with 1M aq NaOH solution (3.94 vol), followed by water (2×4.4 vol), and dried over sodium sulfate (2.0 w/w). The filtrate was concentrated under reduced pressure below 40° C. until no distillate was observed. Tetrahydrofuran (THF) was sequentially added (1×4 vol and 1×6 vol) and concentrated under reduced pressure below 40° C. until no distillate was observed to obtained Compound 1B as light yellow colored syrup liquid.

### Step 3: tert-butyl ((6-(7-chlorothieno[3,2-b]pyridin-2-yl)pyridin-3-yl)methyl)(2-methoxyethyl)carbamate (Compound 1C)

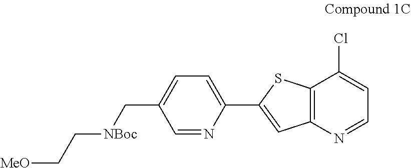 [SMILES: COCCN(Cc1ccc(-c2cc3nccc(Cl)c3s2)nc1)C(=O)OC(C)(C)C]

To a stirred solution of 7-chlorothieno[3,2-b]pyridine (1.05 eq) in tetrahydrofuran (7 vol) was added n-butyl lithium (2.5 M in hexane) drop wise at −15±10° C. and stirred for 90 minutes at the same temperature under nitrogen atmosphere. Zinc chloride (1.05 eq) was added to the reaction mass at −15±10° C. The reaction mass was slowly warmed to 25±5° C. and stirred for 45 minutes under nitrogen atmosphere to afford Compound 1C. The progress of the reaction was monitored by HPLC.

### Step 4: tert-butyl ((6-(7-(4-amino-2-fluorophenoxy)thieno[3,2-b]pyridin-2-yl)pyridin-3-yl)methyl)(2-methoxyethyl)carbamate (Compound 1D)

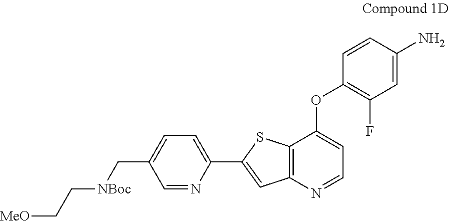 [SMILES: COCCN(Cc1ccc(-c2cc3nccc(Oc4ccc(N)cc4F)c3s2)nc1)C(=O)OC(C)(C)C]

3-fluoro-4-hydroxybenzenaminium chloride (1.2 eq) in DMSO (3.9 vol) at 25±5° C. was charged under nitrogen atmosphere and the reaction mass was stirred until observance of a clear solution at 25±5° C. t-BuOK was added in a lot wise under nitrogen atmosphere at 25±10° C. The reaction mass temperature was raised to 45±5° C. and maintained for 30 minutes under nitrogen atmosphere. Compound 1C was charged lot wise under nitrogen atmosphere at 45±5° C. and stirred for 10 minutes at 45±5° C. The reaction mixture was heated to 100±5° C. and stirred for 2 hrs. The reaction mass is monitored by HPLC.

After reaction completion, the reaction mass was cooled to 10±5° C. and quenched with chilled water (20 vol) at 10±5° C. The mass temperature was raised to 25±5° C. and stirred for 7-8 h. The resulting Compound 1D crude was collected by filtration and washed with 2 vol of water. Crude Compound 1D material was taken in water (10 vol) and stirred for up to 20 minutes at 25±5° C. The reaction mass was heated to 45±5° C. and stirred for 2-3 h at 45±5° C., filtered and vacuum-dried.

Crude Compound 1D was taken in MTBE (5 vol) at 25±5° C. and stirred for about 20 minutes at 25±5° C. The reaction mass temperature was raised to 45±5° C., stirred for 3-4 h at 45±5° C. and then cooled to 20±5° C. The reaction mass was stirred for about 20 minutes at 20±5° C., filtered, followed by bed wash with water (0.5 vol) and vacuum-dried.

The crude material was dissolved in acetone (10 vol) at 25±5° C. and stirred for about 2 h at 25±5° C. The reaction mass was filtered through a celite bed and washed with acetone (2.5 vol). The filtrate was slowly diluted with water (15 vol) at 25±5° C. The reaction mass was stirred for 2-3 h at 25±5° C., filtered and bed washed with water (2 vol) & vacuum-dried to afford Compound 1D as brown solid.

### Step 5: 1-((4-((2-(5-(((tert-butoxycarbonyl)(2-methoxyethyl)amino)methyl)pyridin-2-yl)thieno[3,2-b]pyridin-7-yl)oxy)-3-fluorophenyl)carbamoyl)cyclopropane-1-carboxylic acid (Compound 1E)

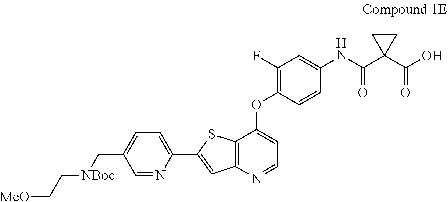 [SMILES: COCCN(Cc1ccc(-c2cc3nccc(Oc4ccc(NC(=O)C5(C(=O)O)CC5)cc4F)c3s2)nc1)C(=O)OC(C)(C)C]

To a solution of Compound 1D (1.0 eq.) in tetrahydrofuran (7 vol.), aqueous potassium carbonate (1.0 eq.) in water (8 vol.) was added. The solution was cooled to 5±5° C., and stirred for about 60 min. While stirring, separately triethylamine (2.0 eq.) was added to a solution of 1,1-cyclopropanedicarboxylic acid (2.0 eq.) in tetrahydrofuran (8 vol.), at 5±5° C., followed by thionyl chloride (2.0 eq.) and stirred for about 60 min. The acid chloride mass was slowly added to the Compound 1D solution at 5±5° C. The temperature was raised to 25±5° C. and stirred for 3.0 h. The reaction was monitored by HPLC analysis.

After reaction completion, the mass was diluted with ethyl acetate (5.8 vol.), water (5.1 vol.), 10% (w/w) aqueous hydrochloric acid solution (0.8 vol.) and 25% (w/w) aqueous sodium chloride solution (2 vol.). The aqueous layer was separated and extracted with ethyl acetate (2×5 vol.). The combined organic layers were washed with a 0.5M aqueous sodium bicarbonate solution (7.5 vol.). The organic layer was treated with Darco activated charcoal (0.5 w/w) and sodium sulfate (0.3 w/w) at 25±5° C. for 1.0 h. The organic layer was filtered through celite and washed with tetrahydrofuran (5.0 vol.). The filtrate was concentrated under vacuum below 50° C. to about 3 vol and co-distilled with ethyl acetate (2×5 vol.) under vacuum below 50° C. up to ˜3.0 vol. The organic layer was cooled to 15±5° C., stirred for about 60 min., filtered, and the solid was washed with ethyl acetate (2.0 vol.). The material was dried under vacuum at 40±5° C. until water content was less than 1% to afford Compound 1E as brown solid.

### Step 6: tert-butyl ((6-(7-(2-fluoro-4-(1-((4-fluorophenyl)carbamoyl)cyclopropane-1-carboxamido)phenoxy)thieno[3,2-b]pyridin-2-yl)pyridin-3-yl)methyl)(2-methoxyethyl)carbamate (Compound 1F)

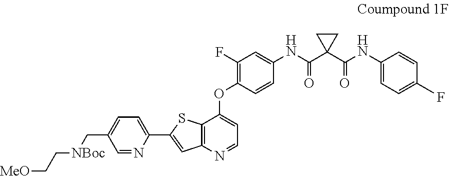 [SMILES: COCCN(Cc1ccc(-c2cc3nccc(Oc4ccc(NC(=O)C5(C(=O)Nc6ccc(F)cc6)CC5)cc4F)c3s2)nc1)C(=O)OC(C)(C)C]

Pyridine (1.1 eq.) was added to a suspension of Compound 1E (1.0 eq.) in tetrahydrofuran (10 vol.) and cooled to 5±5° C. Thionyl chloride (2.0 eq.) was added and stirred for about 60 min. The resulting acid chloride formation was confirmed by HPLC analysis after quenching the sample in methanol. Separately, aqueous potassium carbonate (2.5 eq.) solution (7.0 vol. of water) was added to a solution of 4-fluoroaniline (3.5 eq.) in tetrahydrofuran (10 vol.), cooled to 5±5° C., and stirred for about 60 min. The temperature of the acid chloride mass at 5±5° C. was raised to a temperature of about 25±5° C. and stirred for 3 h. The reaction was monitored by HPLC analysis.

After completion of the reaction, the solution was diluted with ethyl acetate (25 vol.), the organic layer was separated and washed with a 1M aqueous sodium hydroxide solution (7.5 vol.), a 1M aqueous hydrochloric acid solution (7.5 vol.), and a 25% (w/w) aqueous sodium chloride solution (7.5 vol.). The organic layer was dried and filtered with sodium sulfate (1.0 w/w). The filtrate was concentrated ˜3 vol under vacuum below 50° C. and co-distilled with ethyl acetate (3×5 vol.) under vacuum below 50° C. to ˜3.0 vol. Ethyl acetate (5 vol.) and MTBE (10 vol.) were charged, heated up to 50±5° C. and stirred for 30-60 min. The mixture was cooled to 15±5° C., stirred for about 30 min., filtered, and the solid was washed with ethyl acetate (2.0 vol.). MGB3 content was analyzed by HPLC analysis. The material was dried under vacuum at 40±5° C. until the water content reached about 3.0% to afford Compound 1F as brown solid.

### Step 7: N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide (Compound 1)

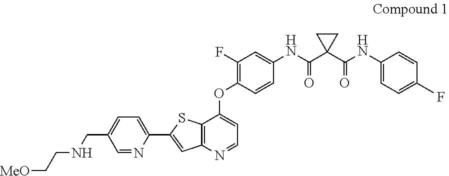 [SMILES: COCCNCc1ccc(-c2cc3nccc(Oc4ccc(NC(=O)C5(C(=O)Nc6ccc(F)cc6)CC5)cc4F)c3s2)nc1]

To a mixture of Compound 1F in glacial acetic acid (3.5 vol.) concentrated hydrochloric acid (0.5 vol.) was added and stirred at 25±5° C. for 1.0 h. The reaction was monitored by HPLC analysis.

After reaction completion, the mass was added to water (11 vol.) and stirred for 20±5° C. for 30 min. The pH was adjusted to 3.0±0.5 using 10% (w/w) aqueous sodium bicarbonate solution and stirred for 20±5° C. for approximately 3.0 h. The mass was filtered, washed with water (4×5.0 vol.) and the pH of the filtrate was checked after every wash. The material was dried under vacuum at 50±5° C. until water content was about 10%.

Crude Compound 1 was taken in ethyl acetate (30 vol.), heated to 70±10° C., stirred for 1.0 h., cooled to 25±5° C., filtered, and washed with ethyl acetate (2 vol.). The material was dried under vacuum at 45±5° C. for 6.0 h.

Crude Compound 1 was taken in polish filtered tetrahydrofuran (30 vol.) and pre-washed Amberlyst® A-21 Ion exchange resin and stirred at 25±5° C. until the solution became clear. After getting a clear solution, the resin was filtered and washed with polish filtered tetrahydrofuran (15 vol.). The filtrate was concentrated by ˜50% under vacuum below 50° C. and co-distilled with polish filtered IPA (3×15.0 vol.) and concentrated up to ˜50% under vacuum below 50° C. Charged polish filtered IPA (15 vol.) was added and the solution concentrated under vacuum below 50° C. to ˜20 vol. The reaction mass was heated to 80±5° C., stirred for 60 min. and cooled to 25±5° C. The resultant reaction mass was stirred for about 20 hours at 25±5° C. The reaction mass was cooled to 0±5° C., stirred for 4-5 hours, filtered, and washed with polish filtered IPA (2 vol.). The material was dried under vacuum at 45±5° C., until the water content was about 2%, to obtain the desired product Compound 1. ¹H-NMR (400 MHz, DMSO-d6): δ10.40 (s, 1H), 10.01 (s, 1H), 8.59-8.55 (m, 1H), 8.53 (d, J=5.6 Hz, 1H), 8.32 (s, 1H), 8.23 (d, J=8.0 Hz, 1H), 7.96-7.86 (m, 2H), 7.70-7.60 (m, 2H), 7.56-7.43 (m, 2H), 7.20-7.11 (m, 2H), 6.66 (d, J=5.6 Hz, 1H), 3.78 (s, 2H), 3.41 (t, J=5.6 Hz, 2H), 3.25 (s, 3H), 2.66 (t, J=5.6 Hz, 2H), 1.48 (s, 4H)ppm. MS: M/e 630 (M+1)⁺.

### Example 1B: Preparation of Compound 1 Crystalline Form D

To a 50 L reactor, 7.15 Kg of Compound 1, 40 g of Form D as crystal seed and 21 L acetone (≥99%) were added. The mixture was heated to reflux (˜56° C.) for 1˜2 h. The mixture was agitated with an internal temperature of 20±5° C. for at least 24 h. Then the suspension was filtered and washed the filter cake with 7 L acetone. The wet cake was dried under vacuum at ≤45° C., to obtain 5.33 kg of Compound 1 of desired Form D.

X-Ray Powder Diffraction (XRPD)

The XRPD patterns were collected with a PANalytical X′ Pert PRO MPD diffractometer using an incident beam of Cu radiation produced using an Optix long, fine-focus source. An elliptically graded multilayer mirror was used to focus Cu Ka X-rays through the specimens and onto the detector. Prior to the analysis, a silicon specimen (NIST SRM 640e) was analyzed to verify the observed position of the Si Ill peak is consistent with the NIST-certified position. A specimen of each sample was sandwiched between 3-μm-thick films and analyzed in transmission geometry. A beam-stop, short antiscatter extension, and an antiscatter knife edge were used to minimize the background generated by air. Soller slits for the incident and diffracted beams were used to minimize broadening from axial divergence. The diffraction patterns were collected using a scanning position-sensitive detector (X′Celerator) located 240 mm from the specimens and Data Collector software v. 2.2b. Pattern Match v2.3.6 was used to create XRPD patterns.

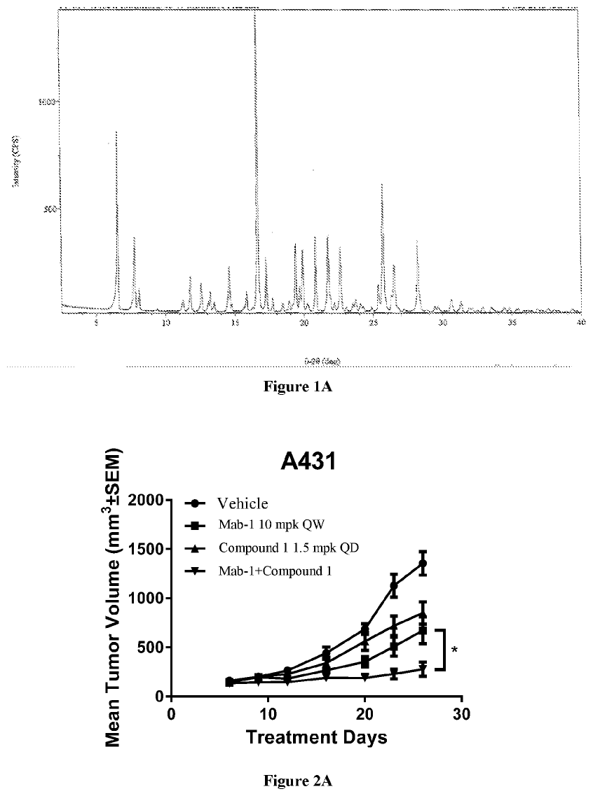

The X-ray powder diffraction (XRPD) pattern was used to characterize the Compound 1 obtained, which showed that the Compound 1 was in Crystalline Form D of Compound 1 (Compound 1 Form D), see FIG. 1A.

### Example 2C: Preparation of Compound 1 Form D

427.0 mg of Compound 1 was dissolved in 5 mL of THF to obtain a clear brown solution. The resulting solution was filtered, and the filtrate evaporated under the flow of nitrogen. A sticky solid was obtained, which was dried under vacuum in room temperature for ˜5 min, still a sticky brown solid obtained. It was dissolved in 0.2 mL of EtOAc and sonicated to dissolve. The solution obtained was stirred at room temperature for 15 min and a solid precipitated. The resulting solid was added 0.4 mL of EtOAc and stirred in room temperature for 21 h 40 min to obtain a suspension. The solid was separated from the mother liquor by centrifugation, then the resulting solid was resuspended in 0.6 mL of EtOAc and stirred at room temperature for 2 days. The solid was isolated by centrifugation, to obtain Compound 1 of desired Form D.

The X-ray powder diffraction (XRPD) pattern was used to characterize the Compound 1 obtained, which showed that Compound 1 was in Crystalline Form D of Compound 1 (Compound 1 Form D).

Crystallization in Example 1 may be done with or without crystal seed. The crystal seed may come from any previous batch of the desired crystalline form. The addition of crystal seed may not affect the preparation of the crystalline forms in the present disclosure.

### Example 2

Combination Efficacy on Multi-Tyrosine Kinase Inhibitor Combined with Anti-PD-1 Antibody

### Example 2A: Study of Compound 1 in Combination with Mab-1 in A431 Allogeneic Model

Method

Female Nod-Scid mice were pretreated with cyclophosphamide (in saline, 100 mg/kg intraperitoneal, QD×2) and disulfiram (in 0.8% Tween-80 in saline 125 mg/kg oral gavage, 2 hr after the dose of cyclophosphamide). Two days later, mice with higher body weight were subcutaneously implanted with 2.5×10⁶ A431 (epidermal carcinoma) cells and 5×10⁶ freshly isolated peripheral blood mononuclear cells (PBMC) on the right flank. After inoculation, mice were randomly assigned into 4 groups with 10 animals in each group according to body weight. Then mice were treated with vehicle (PEG400/0.1N HCL in saline, 40/60), Mab-1, Compound 1, and combination of Mab-1 and Compound 1, respectively. Mab-1 was given at 10 mg/kg once per week (QW) by intraperitoneal (i.p.) injection, and Compound 1 was administered at 1.5 mg/kg once per day (QD) by oral gavage (P.O.). Tumor volume was determined twice weekly in two dimensions using a caliper, and expressed in mm³ using the formula: V=0.5(a×b²), where a and b are the long and short diameters of the tumor, respectively.

Data are presented as mean tumor volume±standard error of the mean (SEM). Tumor growth inhibition (TGI) is calculated using the following formula:

$$\% growth inhibition = 100 ⨯ ( 1 - ( \frac{( treated t ) - ( treated to )}{( placebo t ) - ( placebo to )} ) )$$

treated t=treated tumor volume at time t;treated t₀=treated tumor volume at time 0;
placebo t=placebo tumor volume at time t;
placebo t₀=placebo tumor volume at time 0.

Result

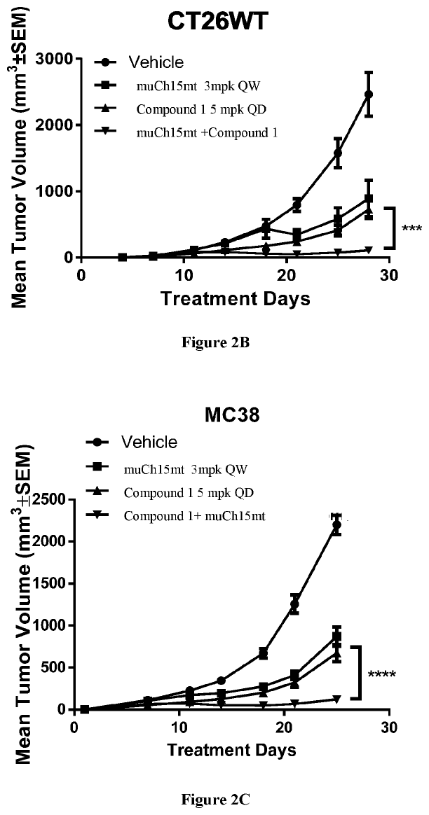

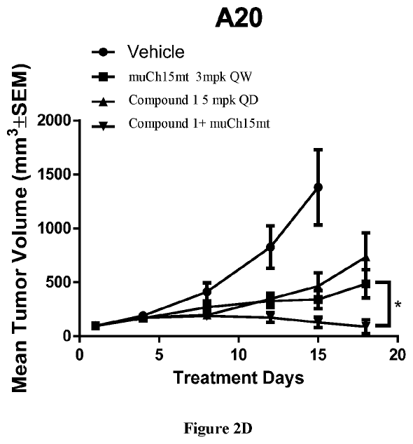

The response of A431 allogeneic xenografts to Mab-1 and Compound 1 was shown in FIG. 2A and Table 2A. On day 26, Mab-1 (10 mg/kg QW×4), Compound 1 (1.5 mg/kg QD×26), and their combination treatment resulted in 50%, 37%, and 79% statistically significant tumor growth inhibition (TGI), respectively, which suggested the strong combination effect of Mab-1 and Compound 1 in A431 allogeneic model. No sign of toxicity was observed during the entire treatment duration.

TABLE 2A
Combination Efficacy of Mab-1 and Compound 1 in
A431 Allogeneic Model

| | | | | Mean Tumor |
|---|---|---|---|---|
| | | | TGI | Volume |
| | Dose | | (%) on | on Day 26 |
| Test Article | (mg/Kg) | N | Day 26 | (mm³) |
| Vehicle | 0 | 8 | — | 1355.5 |
| Mab-1 | 10 | 7 | 50 | 673.3 |
| Compound 1 | 1.5 | 7 | 37 | 851.5 |
| Mab-1 + Compound 1 | 10 + 1.5 | 8 | 79 | 278.3 |

### Example 2B: Study of Compound 1 in Combination with muCh15mt in CT26WT Syngeneic Model

MuCh15mt used herein is an internally generated anti-PD-1 antibody.

Method

Female BALB/c mice were s.c implanted with 2×10⁵ CT26WT (colon carcinoma) cells per 150 ul PBS in the right flank. After inoculation, mice were randomized into 4 groups with 12 animals in each group. The mice were treated with vehicle (PEG400/0.1N HCL in saline, 40/60), muCh15mt, Compound 1, and the combination of muCh15mt and Compound 1, respectively. MuCh15mt was given at 3 mg/kg once per week (QW) by intraperitoneal (i.p.) injection, and Compound 1 was administered at 5 mg/kg once per day (QD) by oral gavage. Tumor volume was determined twice weekly in two dimensions using a caliper, and was expressed in mm³ using the formula: V=0.5(a×b²) where a and b are the long and short diameters of the tumor, respectively. Data are presented as mean tumor volume 1 standard error of the mean (SEM). Tumor growth inhibition (TGI) is calculated using the following formula:

$$\% growth inhibition = 100 ⨯ ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

Result

The response of CT26WT syngeneic model to muCh15mt and Compound 1 was shown in FIG. 2B and Table 2B. On day 28, muCh15mt (3 mg/kg QW×4), Compound 1 (5 mg/kg QD×28) and their combination treatment resulted in 64%, 71%, and 96% tumor growth inhibition, respectively. Meanwhile, the treatments also induced 20%, 6.7%, and 66.7% animals with tumor free, respectively. The data suggested the strong combination anti-tumor activity of Compound 1 and muCh15mt in CT26WT model. No sign of toxicity was observed during the entire treatment duration.

TABLE 2B
Combination Efficacy of muCh15mt and Compound 1 in CT26WT
Syngeneic Model

| | | | Tumor | | Mean |
|---|---|---|---|---|---|
| | | | Free | | Tumor |
| | | | Animal | TGI | Volume |
| | Dose | | (%) on | (%) on | on Day 28 |
| Test Article | (mg/Kg) | N | Day 28 | Day 28 | (mm³) |
| Vehicle | 0 | 15 | 0 | — | 2463.6 |
| muCh15mt | 3 | 15 | 20.0 | 64 | 890.0 |
| Compound 1 | 5 | 15 | 6.7 | 71 | 723.2 |
| muCh15mt + Compound 1 | 3 + 5 | 15 | 66.7 | 96 | 109.3 |

### Example 2C: Study of Compound 1 in Combination with muCh15mt in MC38 Syngeneic Model

Method

Female BALB/C mice were s.c implanted with 1×10⁶ MC38 (colon adenocarcinoma) cells per 150 μl PBS in the right flank. After inoculation, mice were randomized into 4 groups with 15 animals in each group. The mice were treated with vehicle (PEG400/0.1N HCL in saline, 40/60), muCh15mt, Compound 1, and the combination treatment of much15mt and Compound 1, respectively. MuCh15mt was given at 3 mg/kg once per week (QW) by intraperitoneal (i.p.) injection, and Compound 1 was administered at 5 mg/kg once per day (QD) for 25 days by oral gavage (P.O.). Tumor volumes were determined twice weekly in two dimensions using a caliper, and were expressed in mm³ using the formula: V=0.5(a×b²) where a and b are the long and short diameters of the tumor, respectively.

Data are presented as mean tumor volume+standard error of the mean (SEM). Tumor growth inhihition (TGI) is calculated using the following formula:

$$\% growth inhibition = 100 ⨯ ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

Result

The response of MC38 syngeneic model to muCh15mt and Compound 1 was shown in FIG. 2C and Table 2C. On day 25, muCh15mt (3 mg/kg QW×4), Compound 1 (5 mg/kg QD×25) and their combination treatment resulted in 42%, 56% and 96% tumor growth inhibition, respectively. Meanwhile, the combination treatment induced 40% animals with tumor free. The data suggested the strong combination anti-tumor activity of Compound 1 with muCh15mt in MC38 syngeneic model.

TABLE 2C
Combination Efficacy of muCh15mt and Compound 1 in MC38
Syngeneic Model

| | | | Tumor | | Mean |
|---|---|---|---|---|---|
| | | | Free | | Tumor |
| | | | Animal | TGI | Volume |
| | Dose | | % on | (%) on | on Day 25 |
| Test Article | (mg/Kg) | N | Day 25 | Day 25 | (mm³) |
| Vehicle | 0 | 15 | 0 | — | 2198.1 |
| muCh15mt | 3 | 15 | 0 | 42 | 1412.5 |
| Compound 1 | 5 | 15 | 0 | 56 | 1089.5 |
| muCh15mt + Compound 1 | 3 + 5 | 15 | 40.0 | 94 | 122.6 |

### Example 2D: Study of Compound 1 in Combination with muCh15mt in A20 Syngeneic Model

Method

Female BALB/C mice were s.c implanted with 1×10⁵ A20 (B cell lymphoma) cells per 150 μl PBS in the right flank. After inoculation, tumor volumes were determined twice weekly in two dimensions using a caliper, and were expressed in mm³ using the formula: V=0.5(a×b²) where a and b are the long and short diameters of the tumor, respectively. When tumor volume reached approximately 100 mm³, mice were randomized into 4 groups with 12 animals in each group according to tumor volume. The mice were then treated with vehicle (PEG400/0.1N HCL in saline, 40/60), muCh15mt, Compound 1, and the combination, respectively. MuCh15mt was given at 3 mg/kg once per week (QW) by intraperitoneal (i.p.) injection, and Compound 1 was administered at 5 mg/kg once per day (QD) by oral gavage (P.O.).

Data are presented as mean tumor volume ±standard error of the mean (SEM). Tumor growth inhibition (TGI) is calculated using the following formula:

$$\% growth inhibition = 100 ⨯ ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

Result

The response of A20 syngeneic model to muCh15mt and Compound 1 was shown in FIG. 2D and Table 2D. On day 15, muCh15mt (3 mg/kg QW×3), Compound 1 (5 mg/kg QD×18) and their combo treatment resulted in 81%, 71%, and 97% tumor growth inhibition, respectively. Meanwhile, the treatments also induced 33.3%, 8.3%, and 91.7% animals with tumor free, respectively. The data suggested the strong combination anti-tumor activity of Compound 1 with muCh15mt in A20 syngeneic model.

TABLE 2D
Combination Efficacy of muCh15mt and Compound 1 in A20
Syngeneic Model

| | | | Tumor | | Mean |
|---|---|---|---|---|---|
| | | | Free | | Tumor |
| | | | Animal | TGI | Volume |
| | Dose | | % on | (%) on | on Day 18 |
| Test Article | (mg/Kg) | N | Day 18 | Day 15 | (mm³) |
| Vehicle | 0 | 12 | 0 | — | —^{a} |
| muCh15mt | 3 | 12 | 33.3 | 81 | 487.0 |
| Compound 1 | 5 | 12 | 8.3 | 71 | 735.7 |
| muCh15mt + Compound 1 | 3 + 5 | 12 | 91.7 | 97 | 90 |

^{a}data was not presented due to two animals were sacrificed with their tumor volumes >2000 mm³

### Example 3

Clinical Trial Studies on Multi-Tyrosine Kinase Inhibitor Combined with Mab-1

### Example 3A: Clinical Trial Phase I Study

Method

This is an open-label, multicenter, non-randomized Phase 1b clinical trial for patients with histologically or cytologically confirmed locally advanced or metastatic tumors including squamous or non-squamous non-small cell lung cancer (NSCLC), renal cell carcinoma (RCC), ovarian cancer (OC), or melanoma.

All patients will receive Compound 1 120 mg orally once daily (QD) in combination with Mab-1 200 mg IV once every 3 weeks (Q3W) until the occurrence of disease progression (PD), unacceptable toxicity, death, withdrawal of consent, or study termination.

The primary objective of the study was to assess the safety and tolerability of Compound 1 and Mab-1 as a combination therapy. Overall response rate, duration of response (DOR), disease control rate, and progression-free survival (PFS) were assessed as secondary endpoints.

There are 9 cohorts, Cohorts A through I, in the study. Approximately 20 patients will be enrolled into each cohort except Cohort E which expanded up to 60 patients. The patients will be enrolled according to their tumor type and prior anti-programmed cell death protein-1 (PD-1)/PD-L1 antibody treatment.
a) Cohort A: Anti-PD-1/PD-L1 antibody refractory/resistant metastatic, non-squamous NSCLC;
b) Cohort B: Anti-PD-1/PD-L1 antibody naïve metastatic, non-squamous NSCLC;
c) Cohort C: Anti-PD-1/PD-L1 antibody refractory/resistant metastatic or advanced RCC;
d) Cohort D: Metastatic or advanced RCC without prior systemic therapy;
e) Cohort E: Anti-PD-1/PD-L1 antibody naïve recurrent and platinum resistant epithelial OC;
f) Cohort F: Anti-PD-1/PD-L1 antibody refractory/resistant metastatic, squamous NSCLC;
g) Cohort G: Anti-PD-1/PD-L1 antibody refractory/resistant unresectable or metastatic melanoma;
h) Cohort H: positive PD-L1 expression, treatment naïve, locally advanced or metastatic non-squamous NSCLC;
i) Cohort I: positive PD-L1 expression, treatment naïve, locally advanced or metastatic squamous NSCLC.

Criteria

Inclusion Criteria:

1. Able to provide written informed consent and can understand and agree to comply with the requirements of the study and the Schedule of Assessments
2. Age≥18 years on the day of signing the informed consent form (or the legal age of consent in the jurisdiction in which the study is taking place)
3. At least 1 measurable lesion as defined by RECIST v1.1
4. Provide archival tumor tissue (formalin-fixed paraffin-embedded block [FFPE] with tumor tissue or unstained slides), if available.

### 5. Eastern Cooperative Oncology Group (ECOG) Performance Status≤1

6. Adequate hematologic and end-organ function
7. Patients with inactive/asymptomatic carrier, chronic, or active hepatitis B virus (HBV) must have HBV deoxyribonucleic acid (DNA)<500 IU/mL (or 2500 copies/mL) at Screening
8. Females of childbearing potential must be willing to use a highly effective method of birth control for the duration of the study, and ≥120 days after the last dose of study drugs and have a negative serum pregnancy test ≤7 days of the first dose of study drugs
9. Non-sterile males must be willing to use a highly effective method of birth control for the duration of the study and for ≥120 days after the last dose of study drugs

Exclusion Criteria:

1. Unacceptable toxicity on prior anti-PD-1/PD-L1 treatment.
2. Active leptomeningeal disease or uncontrolled brain metastasis.
3. Active autoimmune diseases or a history of autoimmune diseases that may relapse.
4. Any active malignancy 2 years
5. Any condition that required systemic treatment with either corticosteroids (>10 mg daily of prednisone or equivalent) or other immunosuppressive medication ≤14 days before the first dose of study drugs
6. History of interstitial lung disease, noninfectious pneumonitis or uncontrolled diseases, including pulmonary fibrosis, acute lung diseases, etc.
7. 8. Severe chronic or active infections (including tuberculosis infection, etc.) requiring systemic antibacterial, antifungal or antiviral therapy, within 14 days prior to the first dose of study drugs
8. Known history of HIV infection
9. Patients with active hepatitis C infection.
10. Any major surgical procedure requiring general anesthesia ≤28 days before the first dose of study drugs
11. Prior allogeneic stem cell transplantation or organ transplantation
12. Hypersensitivity to tislelizumab or sitravatinib, to any ingredient in the formulation, or to any component of the container
13. Bleeding or thrombotic disorders or use of anticoagulants such as warfarin or similar agents requiring therapeutic INR monitoring within 6 months before the first dose of study drugs
15. Concurrent participation in another therapeutic clinical trial

Conclusions/Results

Combination treatment with Mab-1 and Compound 1 may have promising antitumor activity in non-small cell lung cancer (NSCLC) patients, renal cell carcinoma (RCC) patients, ovarian cancer (OC) patients, or melanoma patients.

As of data cut-off on 17 Jul. 2019, for Cohort E (anti-PD-1/PD-L1 antibody-naive patients with recurrent, platinum-resistant, epithelial OC), 20 patients were enrolled. All 20 patients who received study drugs were included in the safety analysis. Treatment-emergent adverse events (TEAEs) in ≥30% of patients were diarrhea, hypertension, abdominal pain, nausea, fatigue, decreased appetite, and hypomagnesemia. Eight (40.0%) patients experienced grade ≥3 TEAEs related to Compound 1, whereas 2 (10.0%) patients experienced grade ≥3 TEAEs related to Mab-1. Of the 17 efficacy-evaluable patients, 4 patients achieved a partial response, 11 patients had stable disease, and 2 patients had progressive disease per RECIST version 1.1 criteria] Median PFS was 18.0 weeks and median DOR was NR (both ranges, 12.29 weeks-NR). Combination treatment with Mab-1 and Compound 1 was manageable, with promising antitumor activity in ovarian cancer patients.

As of 26 Jun. 2020, a total of 160 subjects have been enrolled and treated. Treatment-related Grade ≥3 AEs were reported in 67 subjects (42%), and those reported in 5 or more subjects (≥3%) were hypertension (16%), ALT increased (7 [4%]), diarrhea (7 [4%]), and stomatitis (5 [3%]). Among the 160 subjects with available safety data, Treatment-related SAEs were reported in 44 subjects (28%), and included diarrhea in 6 subjects (4%), and pneumonia and transaminases increased in 4 subjects (3%) each. All other treatment-related SAEs occurred in 2 or fewer subjects (<2%) overall.

The combination of Mab-1 and Compound 1 has demonstrated generally manageable safety profile and showed promising antitumor activity in patients diagnosed with late stage cancer. This clears the unmet medical needs for checkpoint inhibitor naïve, resistant or refractory patients.

The foregoing examples and description of certain embodiments should be taken as illustrating, rather than as limiting the present disclosure as defined by the claims. As will be readily appreciated, numerous variations and combinations of the features set forth above can be utilized without departing from the present disclosure as set forth in the claims. All such variations are intended to be included within the scope of the present disclosure. All references cited are incorporated herein by reference in their entireties.

1. A method for the prevention, delay of progression or treatment of cancer in a subject, comprising administering to the subject in need thereof a therapeutically effective amount of a multi-tyrosine kinase inhibitor, in combination with a therapeutically effective amount of a PD-1 antagonist,
wherein the multi-tyrosine kinase inhibitor is Compound 1, 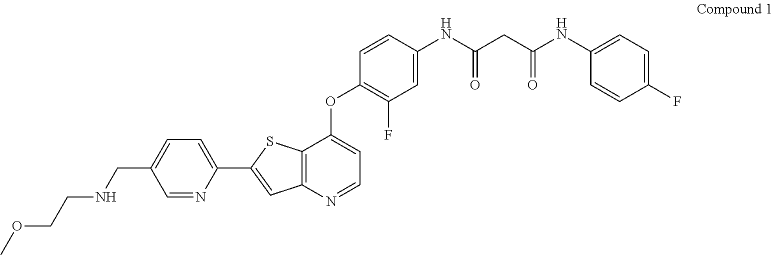 [SMILES: COCCNCc1ccc(-c2cc3nccc(Oc4ccc(NC(=O)CC(=O)Nc5ccc(F)cc5)cc4F)c3s2)nc1]
or a stereoisomer thereof, or a pharmaceutically acceptable salt thereof,
wherein the PD-1 antagonist is an antibody or a fragment antigen binding thereof, which specifically binds to human PD-1 comprising a heavy chain variable region (Vh) and a light chain variable region (Vk) that contain complement determinant regions (CDRs) listed as follows:
a) mu317 CDR-H1, CDR-H2 and CDR-H3 (SEQ ID NOs: 11, 12, 13, respectively); and
CDR-L1, CDR-L2 and CDR-L3 (SEQ ID NOs: 14, 15, 16, respectively);
b) mu326 CDR-H1, CDR-H2 and CDR-H3 (SEQ ID NOs: 17, 18, 19, respectively); and
CDR-L1, CDR-L2 and CDR-L3 (SEQ ID NOs: 20, 21, 22, respectively);
c) 317-4B6 CDR-H1, CDR-H2 and CDR-H3 (SEQ ID NOs: 31, 32, 33, respectively); and
CDR-L1, CDR-L2 and CDR-L3 (SEQ ID NOs: 34, 35, 36, respectively);
d) 326-4A3 CDR-H1, CDR-H2 and CDR-H3 (SEQ ID NOs: 37, 38, 39, respectively); and
CDR-L1, CDR-L2 and CDR-L3 (SEQ ID NOs: 40, 41, 42, respectively);
e) 317-1H CDR-H1, CDR-H2 and CDR-H3 (SEQ ID NOs: 11, 59, 13, respectively); and
CDR-L1, CDR-L2 and CDR-L3 (SEQ ID NOs: 14, 15, 16, respectively);
f) 317-4B2 CDR-HL CDR-H2 and CDR-H3 (SEQ ID NOs: 11, 60, 13, respectively); and
CDR-L1, CDR-L2 and CDR-L3 (SEQ ID NOs: 61, 15, 16, respectively);
g) 317-4B5 CDR-H1, CDR-H2 and CDR-H3 (SEQ ID NOs: 11, 60, 13, respectively); and
CDR-L1, CDR-L2 and CDR-L3 (SEQ ID NOs: 61, 15, 16, respectively);
h) 317-4B6 CDR-H1, CDR-H2 and CDR-H3 (SEQ ID NOs: 11, 32, 13, respectively); and
CDR-L1, CDR-L2 and CDR-L3 (SEQ ID NOs: 61, 15, 16, respectively);
i) 326-1 CDR-H1, CDR-H2 and CDR-H3 (SEQ ID NOs: 17, 62, 19, respectively); and
CDR-L1, CDR-L2 and CDR-L3 (SEQ ID NOs: 20, 21, 22, respectively);
j) 326-3B1 CDR-H1, CDR-H2 and CDR-H3 (SEQ ID NOs: 17, 62, 19, respectively); and
CDR-L1, CDR-L2 and CDR-L3 (SEQ ID NOs: 20, 21, 22, respectively); or
k) 326-3G1 CDR-H1, CDR-H2 and CDR-H3 (SEQ ID NOs: 17, 62, 19, respectively); and
CDR-L1, CDR-1 2 and CDR-L3 (SEQ ID NOs: 20, 21, 22, respectively).

2. The method of claim 1, wherein the PD-1 antagonist is an antibody which comprises a heavy chain variable region (Vh) and a light chain variable region (Vk) comprising:
a) mu317 (SEQ ID NOs: 4 and 6, respectively); p) 317-3H1 (SEQ ID NOs: 69 and 26, respectively);
b) mu326 (SEQ ID NOs: 8 and 10, respectively); q) 317-311 (SEQ ID NOs: 70 and 26, respectively);
c) 317-4B6 (SEQ ID NOs: 24 and 26, respectively);
d) 326-4A3 (SEQ ID NOs: 28 and 30, respectively); r) 317-4B 1 (SEQ ID NOs: 71 and 26, respectively);
e) 317-4B2 (SEQ ID NOs: 43 and 44, respectively); s) 317-4B3 (SEQ ID NOs: 72 and 26, respectively);
f) 317-4B5 (SEQ ID NOs: 45 and 46, respectively); t) 317-4B4 (SEQ ID NOs: 73 and 26, respectively);
g) 317-1 (SEQ ID NOs: 48 and 50, respectively); u) 317-4A2 (SEQ ID NOs: 74 and 26, respectively);
h) 326-3B1 (SEQ ID NOs: 51 and 52, respectively); v) 326-3 A 1 (SEQ ID NOs: 75 and 30, respectively);
i) 326-3GI (SEQ ID NOs: 53 and 54, respectively); w) 326-3C (SEQ ID NOs: 76 and 30, respectively);
j) 326-1 (SEQ ID NOs: 56 and 58, respectively); x) 326-3D1 (SEQ ID NOs: 77 and 30, respectively);
k) 317-3A1 (SEQ ID NOs: 64 and 26, respectively); y) 326-3E1 (SEQ ID NOs: 78 and 30, respectively);
1) 317-3C (SEQ ID NOs: 65 and 26, respectively); z) 326-3F1 (SEQ ID NOs: 79 and 30, respectively);
m) 317-3E1 (SEQ ID NOs: 66 and 26, respectively); aa) 326-3B N55D (SEQ ID NOs: 80 and 30, respectively);
n) 317-3F1 (SEQ ID NOs: 67 and 26, respectively); ab) 326-4A1 (SEQ ID NOs: 28 and 81, respectively); or
o) 317-3G1 (SEQ ID NOs: 68 and 26, respectively); ac) 326-4A2 (SEQ ID NOs: 28 and 82, respectively).

3. The method of claim 1, wherein the PD-1 antagonist is an antibody which contains an IgG4 heavy chain effector or constant domain comprising any of SEQ ID NOs: 83-88.

4. The method of claim 1, wherein the PD-1 antagonist is an antibody which contains an F(ab) or F(ab)2 comprising a domain of claim 2.

5. The method according to claim 1, wherein the PD-1 antagonist is an antibody which comprises a heavy chain variable region (Vh) and a light chain variable region (Vk), and a IgG4 heavy chain effector or constant domain comprising SEQ ID NOs: 87 or 88, wherein the heavy chain variable region (Vh) and the light chain variable region (Vk) comprise:
a) mu317 (SEQ ID NOs: 4 and 6, respectively); p) 317-3H1 (SEQ ID NOs: 69 and 26, respectively);
b) mu326 (SEQ ID NOs: 8 and 10, respectively); q) 317-311 (SEQ ID NOs: 70 and 26, respectively);
c) 317-4B6 (SEQ ID NOs: 24 and 26, respectively);
d) 326-4A3 (SEQ ID NOs: 28 and 30, respectively); r) 317-4B 1 (SEQ ID NOs: 71 and 26, respectively);
e) 317-4B2 (SEQ ID NOs: 43 and 44, respectively); s) 317-4B3 (SEQ ID NOs: 72 and 26, respectively);
f) 317-4B5 (SEQ ID NOs: 45 and 46, respectively); t) 317-4B4 (SEQ ID NOs: 73 and 26, respectively);
g) 317-1 (SEQ ID NOs: 48 and 50, respectively); u) 317-4A2 (SEQ ID NOs: 74 and 26, respectively);
h) 326-3B1 (SEQ ID NOs: 51 and 52, respectively); v) 326-3 A 1 (SEQ ID NOs: 75 and 30, respectively);
i) 326-3GI (SEQ ID NOs: 53 and 54, respectively); w) 326-3C1 (SEQ ID NOs: 76 and 30, respectively);
j) 326-1 (SEQ ID NOs: 56 and 58, respectively); x) 326-3D1 (SEQ ID NOs: 77 and 30, respectively);
k) 317-3A1 (SEQ ID NOs: 64 and 26, respectively); y) 326-3E1 (SEQ ID NOs: 78 and 30, respectively);
1) 317-3C (SEQ ID NOs: 65 and 26, respectively); z) 326-3F1 (SEQ ID NOs: 79 and 30, respectively);
m) 317-3E1 (SEQ ID NOs: 66 and 26, respectively); aa) 326-3B N55D (SEQ ID NOs: 80 and 30,
n) 317-3F1 (SEQ ID NOs: 67 and 26, respectively); respectively);
o) 317-3G1 (SEQ ID NOs: 68 and 26, respectively); ab) 326-4A1 (SEQ ID NOs: 28 and 81, respectively); or
ac) 326-4A2 (SEQ ID NOs: 28 and 82, respectively).

6. The method according to claim 1, wherein the PD-1 antagonist is an antibody which comprises a heavy chain variable region (Vh) and a light chain variable region (Vk), and an IgG4 heavy chain effector or constant domain comprising SEQ ID NO: 88, wherein the heavy chain variable region (Vh) and the light chain variable region (Vk) comprises SEQ ID NO: 24 and SEQ ID NO: 26, respectively.

7. The method of claim 1, wherein Compound 1 is in Crystalline Form D.

8. The method of claim 7, wherein the Crystalline Form D has an XRPD pattern substantially as shown in FIG. 1A.

9. The method of claim 1, wherein the cancer is a solid cancer or tumor.

10. The method of claim 9, wherein the cancer is selected from lung cancer including non-small cell lung cancer (NSCLC), ovarian cancer (OC), renal cell carcinoma (RCC) or melanoma.

11. The method of claim 9, wherein cancer is multi-tyrosine kinase-associated cancer.

12. The method of claim 9, the cancer is resistant or refractory to a checkpoint inhibitor selected from anti-PD-1 antibody and/or anti-PD-L1 antibody.

13. The method of claim 9, the cancer is PD-L1 expression.

14. The method of claim 9, the cancer is selected by its stage including locally advanced, recurrent or metastatic.

15. The method of claim 10, wherein the cancer is non-squamous non-small cell lung cancer (NSCLC), squamous non-small cell lung cancer (NSCLC), epithelial ovarian cancer (OC), renal cell carcinoma (RCC) or melanoma.

16. The method of claim 15, wherein the non-squamous non-small cell lung cancer (NSCLC) is: anti-PD-1/PD-L1 antibody refractory/resistant metastatic, non-squamous NSCLC; anti-PD-1/PD-L1 antibody naïve metastatic, non-squamous NSCLC; or, PD-L1 positive, locally advanced or metastatic, non-squamous NSCLC without prior systemic treatment in metastatic setting.

17. The method of claim 15, wherein the squamous non-small cell lung cancer (NSCLC) is: anti-PD-1/PD-L1 antibody refractory/resistant metastatic, squamous NSCLC; anti-PD-1/PD-L1 antibody naïve metastatic, squamous NSCLC; or, PD-L1 positive, locally advanced or metastatic, squamous NSCLC without prior systemic treatment in metastatic setting.

18. The method of claim 15, wherein the renal cell carcinoma (RCC) is anti-PD-1/PD-L1 antibody refractory/resistant metastatic or advanced RCC, or metastatic or advanced RCC without prior systemic therapy.

19. The method of claim 15, wherein the melanoma is anti-PD-1/PD-L1 antibody refractory/resistant unresectable or metastatic melanoma.

20. The method of claim 15, wherein the ovarian cancer (OC) is naïve recurrent and platinum-resistant epithelial OC.

21. The method of any one of claims 1-6, wherein the PD-1 antagonist is administered parenterally at a dose of 30-300 mg QW, or Q2W, or Q3W, or Q4W.

22. The method of any one of claims 1-6, wherein the PD-1 antagonist is administered parenterally at a dose of 200 mg Q3W.

23. The method of any one of claims 1-7, wherein Compound 1 is administered orally at a dose of 40-150 mg QD.

24. The method of any one of claims 1-7, wherein Compound 1 is administered orally at a dose of 60-150 mg QD.

25. The method of any one of claims 1-7, wherein Compound 1 is administered orally at a dose of 120 mg QD.

26. The method of any one of claims 1-7, wherein Compound 1 is administered orally at a dose of 120 mg QD in combination with the PD-1 antagonist at a dose of 200 mg IV Q3W.

27. A pharmaceutical combination for use in the prevention, delay of progression or treatment of cancer, comprising a PD-1 antagonist and a multi-tyrosine kinase inhibitor, wherein the PD-1 antagonist is an antibody or a fragment antigen binding thereof, which specifically binds to human PD-1, comprising a heavy chain variable region (Vh) and a light chain variable region (Vk) that contain complement determinant regions (CDRs) listed as follows:
CDR-H1, CDR-H2 and CDR-H3 (SEQ ID NOs: 31, 32, 33, respectively); and
CDR-L1, CDR-L2 and CDR-L3 (SEQ ID NOs: 34, 35, 36, respectively); and
wherein the multi-tyrosine kinase inhibitor is Compound 1, 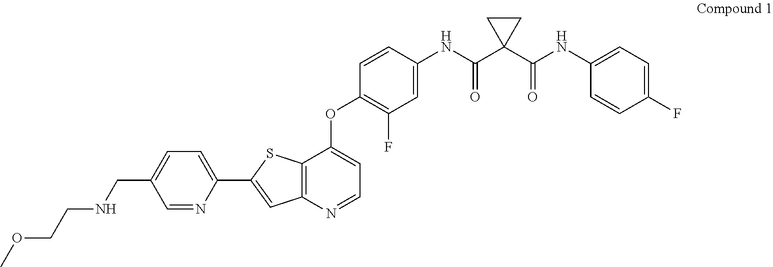 [SMILES: COCCNCc1ccc(-c2cc3nccc(Oc4ccc(NC(=O)C5(C(=O)Nc6ccc(F)cc6)CC5)cc4F)c3s2)nc1]
or a stereoisomer thereof, or a pharmaceutically acceptable salt thereof.
